(19) United States
(12) Patent Application Publication
(10) Pub. No.: US 2014/0228315 A1
(43) Pub. Date: Aug. 14, 2014

(54) BLOCKADE OF EOSINOPHIL PRODUCTION BY TOLL-LIKE RECEPTORS

(71) Applicant: CHILDREN'S HOSPITAL MEDICAL CENTER, Cincinnati, OH (US)

(72) Inventors: Marc E. Rothenberg, Cincinnati, OH (US); Patricia C. Fulkerson, Cincinnati, OH (US)

(21) Appl. No.: 14/126,244

(22) PCT Filed: Jun. 18, 2012
(86) PCT No.: PCT/US2012/042985

§ 371 Date: Apr. 17, 2014

Related U.S. Application Data
(60) Provisional application No. 61/497,796, filed on Jun. 16, 2011.

(51) Int. Cl. A61K 31/739

(52) U.S. Cl. CPC A61K 31/739
USPC 514/54

(57) ABSTRACT

It has long been known that eosinopenia is observed during acute bacterial infection yet the mechanism remains undefined. Herein, we investigated the consequence of exposure to microbial products, specfically bacterial lipopolysaccharide (LPS), on eosinophil production. We demonstrate that developing murine eosinophils transiently express mRNA for six Toll-like receptors (TLR5) with highest expression of TLR2 and TLR4 throughout eosinophil development and nearly undetectable levels on mature eosinophils. LPS stimulation of eosinophil progenitors ex vivo markedly inhibited IL-5- mediated cellular proliferation and expansion Further LPS adrninistratwn in vivo reduced numbers of eosinophil progenitors in the bone marrow and blood in mice. Notably, LPS effectively reduced eosinophilia even in hypereosinophilic mice induced by the IL-S transgene. Taken together, these findings identify a mechanistic explanation for eosinopenia following bacterial infections and a novel therapeutic strategy for depleting eosinophil progenitors and inhibiting peripheral eosinophilia in eosinophil associated diseases.

### CROSS-REFERENCE TO RELATED APPLICATION

The present application claims the benefit of priority under 35 U.S.C. §119(e) to U.S. Provisional Application No. 61/497,796, NEGATIVE REGULATION OF EOSINOPHIL PRODUCTION BY TOLL-LIKE RECEPTORS, filed on Jun. 16, 2011, which is incorporated by reference in its entirety.

### STATEMENT REGARDING FEDERALLY-SPONSORED RESEARCH

This invention was made with U.S. Government support on behalf the National Institute of Health (NIH) Grant Nos. R37 AI045898, R01 AI083450, and K08 AI093673. The U.S. Government has certain rights in this invention.

### FIELD OF THE INVENTION

The invention disclosed herein generally relates to treatment of eosinophilia-associated conditions by suppression of eosinophil progenitor cells.

### BACKGROUND

Eosinophilia, which is characterized by a marked increase in eosinophil counts, is a common clinical problem associated with numerous disorders, such as atopic disease and parasitic infection. While a considerable amount of research has been focused on understanding mechanisms of eosinophil production, particularly those that regulate IL-5-driven eosinophilopoiesis and mature eosinophil survival (Bochner, et al. J. Allergy Clin. Immunol. 126:16-25 (2010)), relatively little is known about endogenous pathways that reduce eosinophil levels even though a marked decrease in circulating eosinophils, or eosinopenia, has long been associated with acute bacterial infections (Bass J. Clin. Invest. 56:870-9 (1975)).

Studies in the 1970s demonstrated that the eosinopenia associated with acute bacterial infection occurred independently of endogenous glucocorticoids (Bass J. Clin. Invest. 55:1229-36 (1975)), but little progress has been made in the subsequent decades to further elucidate the mechanism. Rather, most studies have heretofore focused on delineating the mediators that promote eosinophil accumulation in the blood and tissue, with resultant clinical studies aimed at blocking the effects of IL-5 and related mediators in eosinophil-associated disorders (Stein, et al. Recent Pat. Inflamm. Allergy Drug Discov. 4:201-9 (2010); Busse, et al. J. Allergy Clin. Immunol. 125:803-13 (2010)). The mechanism of infection-associated eosinopenia and its potential for suppressing IL-5-mediated eosinophilia has heretofore not been extensively investigated.

### SUMMARY OF THE INVENTION

Methods and compositions described herein are provided by way of example and should not in any way limit the scope of the invention.

Embodiments of the invention encompass methods of treating an eosinophilia-associated condition in a subject in need thereof, including identifying a subject with an eosinophilia-associated condition and administering to the subject a suppressor of eosinophil progenitor (EoP) proliferation, wherein administration of the suppressor can result in treatment of the eosinophilia-associated condition.

In some embodiments of the methods, the suppressor can interact with a Toll-like receptor. In some embodiments, the Toll-like receptor can be at least one of TLR-4, a TLR-2 heterodimer, or TLR-7, or the like.

In some embodiments, the Toll-like receptor can be TLR-4, and the suppressor can include, for example, a lipopolysaccharide (LPS), monophosphoryl lipid A (MPLA), mannan, a phospholipid, MMTV, RSV, ultrapure MPLA, synthetic MPLA (sMPLA), HSP-22, HSP-60, HSP-70, HSP-96, fibrinogen (extra domain A), minimally modified low-density lipoprotein, and surfactant protein A, and the like. In some embodiments, the Toll-like receptor can be a TLR-2 heterodimer, and the suppressor can include, for example, Pam2CSK4, Pam3CSK4, lipoteichoic acid, zymosan, a porin, MALP2, a bacterial peptidoglycan, lipoarabinomannan, HSP-60, HSP-70, HSP-96, and HMGB1, and the like. In some embodiments, the Toll-like receptor can be TLR-7, and the suppressor can include, for example, CL264, imiquimod, a viral ssRNA, a GU-rich oligoribonucleotide, loxoribin, resiquimod, an adenosine derivative a guanosine derivative, and an immune-complex ssRNA, and the like.

In some embodiments, the suppressor can include, for example, a lipopolysaccharide (LPS), monophosphoryl lipid A (MPLA), mannan, a phospholipid, MMTV, RSV, ultrapure MPLA, synthetic MPLA (sMPLA), HSP-22, HSP-60, HSP-70, HSP-96, fibrinogen (extra domain A), minimally modified low-density lipoprotein, surfactant protein A, Pam2CSK4, Pam3CSK4, lipoteichoic acid, zymosan, a porin, MALP2, a bacterial peptidoglycan, lipoarabinomannan, HSP-60, HSP-70, HSP-96, HMGB1, CL264, imiquimod, a viral ssRNA, a GU-rich oligoribonucleotide, loxoribin, resiquimod, an adenosine derivative a guanosine derivative, and an immune-complex ssRNA, and the like.

In some embodiments, the eosinophilia-associated condition can include, for example, an eosinophil-associated gastrointestinal disorder, eosinophilic pneumonia, allergies, asthma, atopic dermatitis, drug hypersensitivity, eosinophilic leukemia, Churg-Strauss syndrome, and hypereosinophilic syndrome, and the like.

In some embodiments, administration of the suppressor can result in reduced eosinophil production. In some embodiments, administration of the suppressor can result in reduced peripheral eosinophilia.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A-E depict the ex vivo liquid culture system for murine eosinophils. FIG. 1A depicts a schematic of the eosinophil liquid culture system. FIG. 1B depicts the percentage of CD34-expressing cells in the cultures before and after stimulation with SCF and FLT3L. FIG. 1C depicts representative cytospin preparations of LDBM cells harvested from cultures. FIG. 1D depicts the percentage of LDBM cells expressing surface Siglec-F (circles) or CCR3 and Siglec-F (triangles) in the cultures. FIG. 1E depicts the fold changes in normalized mRNA levels for major basic protein (MBP; circles), eosinophil peroxidase (EPX; squares), eosinophil ribonuclease 1 and 2 (EAR1/2; triangles), and myeloperoxidase (MPO; inverted triangles) in cultured LDBM cells.

FIGS. 2A-F depict results demonstrating that TLR4 is expressed by developing eosinophils but not by mature eosinophils. FIG. 2A depicts the fold changes in mRNA levels in LDBM cells cultured in IL-5 as compared to mRNA levels before IL-5 stimulation. FIG. 2B depicts the normalized expression of TLR4 mRNA in LDBM cells cultured in IL-5. FIG. 2C depicts the surface expression of TLR4 on developing eosinophils (CD34⁺) or on mature eosinophils (CCR3⁺) after 4 and 10 days of IL-5 stimulation, respectively. FIG. 2D depicts the percentages of cultured cells co-expressing TLR4 with a marker for the progenitor population (CD34) or for a mature eosinophil (CCR3) after 4 or 10 days of IL-5 stimulation, respectively. FIG. 2E depicts the surface expression of CD34 and TLR4 on freshly isolated wild-type LDBM cells. FIG. 2F depicts the viability and surface expression of CCR3 and Siglec-F at day 14 on TLR4⁺ cells that were isolated on day 8 and cultured for another 6 days in IL-5.

FIGS. 3A-F depict results demonstrating that TLR4 stimulation inhibits IL-5-mediated eosinophil production. FIG. 3A depicts a schematic of LPS exposure for eosinophil progenitors. FIG. 3B depicts eosinophil yields at day 11 and day 15 with and without LPS stimulation. FIG. 3C depicts representative forward and side scatter plots of day 15 cultured cells and a histogram of Siglec-F surface expression of gated cells with and without LPS stimulation. FIG. 3D depicts eosinophil yields at day 15 with and without LPS or ultrapure (UP-) LPS stimulation. FIG. 3E depicts wild-type (WT, solid bars) and TLR4-deficient (TLR4KO, open bars) eosinophil yields at day 14 with and without UP-LPS exposure. FIG. 3F depicts eosinophil yields at day 14 following UP-LPS (open bars), derived monophosphoryl lipid A (MPLA, black bars), or synthetic monophosphoryl lipid A (sMPLA, shaded bars) exposure.

FIGS. 4A-C depict results demonstrating that TLR2 and TLR7 activation inhibits eosinophil production. FIG. 4A depicts eosinophil yield at day 14 following exposure to TLR2 ligands. FIG. 4B depicts eosinophil yield at day 14 following exposure to TLR7 ligands. FIG. 4C depicts eosinophil yield at day 14 following exposure to TLR3 ligand (I, Poly(I:C)) or UP-LPS.

FIGS. 5A-D depict results demonstrating that LPS exposure stimulates production of pro-inflammatory cytokines in eosinophil cultures. FIG. 5A depicts concentrations of IL-6, IL-10, IFN-γ, and TNF-α in culture supernatants 3 and 6 days after LPS stimulation at indicated doses. FIG. 5B depicts eosinophil yields at day 14 from cultures with IL-Sonly or with IL-5 and the indicated cytokines. FIG. 5C depicts eosinophil yields at day 15 from cultures with IL-5 only or with IL-5 with a mixture (MIX) of cytokines (IL-5, IL-6, IL-10, IFN-γ, and TNF-α) for the final 6 days of the culture. FIG. 5D depicts eosinophil yields at day 15 from cultures without LPS exposure (IL-5 only), exposed to LPS, or exposed to LPS with a neutralizing anti-IFN-γ antibody or its isotype control antibody.

FIG. 6A-B depict results demonstrating that LPS exposure inhibits progenitor cell proliferation but does not impair viability. FIG. 6A depicts the percentage of Siglec-F⁺EdU⁺ cells at day 14 after cells were treated with IL-5 only or with IL-5 and LPS (LPS) for 24 hours, then pulsed with EdU for an additional 24 hours. Thereafter, cells were cultured for an additional 3-4 days with IL-5 alone. FIG. 6B depicts the percentages of viable cells in cultures with IL-5 only (squares), LPS with IL-5 (triangles), or with media only with no added cytokines (circles) after 1, 6, and 24 hours starting with day 8 cultured cells.

FIG. 7A-E depict the reduced numbers of EoP colonies and blood eosinophils as a result of endotoxemia. FIG. 7A depicts eosinophil (Eos) and mixed myeloid colonies (mean SD) formed from LDBM cells harvested from wild-type mice 24 hours after intraperitoneal (IP) treatment with saline, 40 mg LPS (LPS-40), or 80 mg LPS (LPS-80). FIG. 7B depicts eosinophil colonies (mean SD) formed from LDBM cells harvested from wild-type mice 8 days after IP with saline or LPS-40. FIG. 7C depicts eosinophil colonies (mean SD) formed from LDBM cells harvested from wild-type mice 24 hours after IP with saline, 50 mg of ultrapure LPS (UP-LPS-50), 50 mg of MPLA (MPLA-50), or 50 or 70 mg synthetic MPLA (sMPLA-50 or sMPLA-70). FIG. 7D depicts eosinophil colonies formed from LDBM cells harvested from IL-5 transgenic mice 24 hours after IP with saline or 10 mg, 25 mg, or 40 mg of LPS (LPS-10, LPS-25, LPS-40). FIG. 7E depicts percentages of blood eosinophils in whole blood from IL-5 transgenic mice 4 days after IP with saline or 40 mg of LPS (LPS-40).

FIGS. 8A-C depict the ability of LPS to inhibit the production of human eosinophils from cord blood progenitors. FIG. 8A depicts a schematic of the human eosinophil liquid culture system. FIG. 8B depicts a representative histogram showing TLR4 expression on progenitors after 6 days of SCF and FLT3L stimulation (n=2 experiments). FIG. 8C depicts eosinophil yields at day 21 with and without LPS (1 mg/mL) stimulation.

FIG. 9 depicts a representation of the regulation of eosinophilia and the local environment by EoPs.

### DETAILED DESCRIPTION OF THE INVENTION

Unless otherwise noted, terms are to be understood according to conventional usage by those of ordinary skill in the relevant art.

All references cited herein are incorporated by reference in their entirety as though fully set forth.

Suppression of parasite-associated eosinophilia with acute bacterial infection in guinea pigs was first noted in 1934. Since that finding, eosinopenia with acute infection has been assumed to be secondary to the release of adrenal glucocorticoids in response to the stress of infection (Bass J. Clin. Invest. 56:870-9 (1975)). However, in a series of elegant murine studies in 1975, the suppression of trichinosis-associated eosinophilia in mice with acute bacterial infection was demonstrated to occur independently of adrenal stimulation in animals after adrenalectomy (Bass J. Clin. Invest. 55:1229-36 (1975)). Follow-up studies published that same year suggested a decline in eosinophil production with acute infection in both resting and stimulated bone marrow (Bass J. Clin. Invest. 56:870-9 (1975)); the decline in eosinophil production was speculated to be due to a direct effect on dividing eosinophils or an effect on the “unrecognizable eosinophil precursor.” Although eosinopenia has long been observed during acute bacterial infection, suggesting the existence of an endogenous pathway for reducing eosinophils, the mechanism of eosinophil reduction has heretofore been poorly understood.

Hematopoietic stem cells (HSCs) give rise to common myeloid progenitors (CMPs) and granulocyte/macrophage progenitors (GMPs), which in turn give rise to eosinophils, neutrophils and macrophages (Mori, et al. J. Exp. Med. 206:183-93 (2009); Iwasaki, et al. J. Exp. Med. 201:1891-97 (2005)). In human hematopoiesis, CMPs give rise to a CD34⁺IL-5Rα⁺ eosinophil progenitor (EoP) (Mori, et al. J. Exp. Med. 206:183-93 (2009)); this CD34⁺IL-5Rα⁺ EoP is increased in a variety of diseases, including atopy, helminth infections, hypereosinophilic syndrome (HES), and select malignancies (Saito, et al. J. Immunol. 168:3017-23 (2002); Sehmi, et al. J. Clin. Invest. 100:2466-75 (1997)). While increased EoP production is an important checkpoint in disease-associated eosinophilia, positive and negative regulation of EoP production has been heretofore poorly understood.

Toll-like receptors (TLRs) are membrane glycoproteins composed of signaling and antigen-recognition domains (Kanzler, et al. Nat. Med. 13:552-9 (2007)). HSCs, CMPs, and GMPs have been shown to express functional TLRs, including TLR2 and TLR4 (Nagai, et al. Immunity 24:801-12 (2006)). Murine eosinophil precursors express mRNA for six TLRs, with high expression of TLR4 throughout eosinophil development. While a number of studies support a role for TLRs in influencing hematopoiesis in a host-protective manner (Nagai, et al. Immunity 24:801-12 (2006); Ueda, et al. J. Exp. Med. 201:1771-80 (2005); McGettrick et al. Br. J. Haematol. 139:185-93 (2007)), no study has heretofore examined the consequence of TLR activation on eosinophil production in the bone marrow.

As described herein, the functional TLR2 heterodimers, TLR4, and TLR7 are expressed by EoPs, thereby demonstrating the consequences of TLR activation on eosinophil production. The role of certain TLRs, including TLR2, TLR3, TLR4, and TLR7, in regulating eosinophil production has been elucidated. The in vitro and in vivo evidence described herein demonstrate a direct inhibitory effect on EoPs by ligands for TLR4, as well as ligands for TLR2 heterdimers and TLR7, as a mechanism for the eosinopenia associated with systemic infections.

Utilizing an ex vivo eosinophil development system and an in vivo transgenic model of IL-5-driven hypereosinophilia, EoPs were found to have a marked sensitivity to TLR ligands that impairs their proliferation. Ligands for TLR2 heterodimers, TLR4, and TLR7, but not TLR3, inhibit eosinophil growth ex vivo by attenuating EoP cell proliferation. Further, in vivo administration of a TLR4 ligand, such as LPS or monophosphoryl lipid A, reduces the number of EoPs in wild-type, but not in TLR4 gene-deficient mice.

As further described herein, LPS dose-dependently reduces hypereosinophilia in IL-5 transgenic mice, demonstrating the therapeutic value of this approach even in an extreme IL-5-driven clinical state. Suppression of eosinophil production by TLR4 ligands was conserved between mice and humans, as yield of eosinophils from human EoPs was reduced following LPS exposure. The results described herein therefore explain eosinopenia following bacterial infections mechanistically and provide a therapeutic strategy for inhibiting eosinophil production and peripheral eosinophilia in eosinophil-associated diseases.

These data support an important role for microbial components recognized by TLRs in negatively regulating eosinophil production in the bone marrow. Thus, the emerging class of TLR agonists have therapeutic potential in reducing eosinophilia in eosinophil-associated disorders (Hennessy, et al. Nat. Rev. Drug Discov. 9:293-307 (2010); Wolska, et al. Curr. Mol. Med. 9:324-35 (2009)).

Embodiments of the invention as disclosed herein relate to methods of treating eosinophilia-associated conditions in a subject by administration of a suppressor of EoP proliferation.

As used herein, the term “treatment” is used in some embodiments to refer to administration of a compound of the present invention to mitigate a disease or a disorder in a host, preferably in a mammalian subject, more preferably in humans. Thus, the term “treatment” can include includes: preventing a disorder from occurring in a host, particularly when the host is predisposed to acquiring the disease, but has not yet been diagnosed with the disease; inhibiting the disorder; and/or alleviating or reversing the disorder. Insofar as the methods of the present invention are directed to preventing disorders, it is understood that the term “prevent” does not require that the disease state be completely thwarted (see Webster's Ninth Collegiate Dictionary). Rather, as used herein, the term preventing refers to the ability of the skilled artisan to identify a population that is susceptible to disorders, such that administration of the compounds of the present invention can occur prior to onset of a disease. The term does not mean that the disease state must be completely avoided.

As used herein, an “eosinophilia-associated condition” or “eosinophilia-associated disease” can refer to any condition that features an enhanced level of eosinophils or their activation state or a disease with clinical or pathological features caused by eosinophils, at least in part. Such conditions include, but are not limited to, eosinophil-associated gastrointestinal disorder, eosinophilic esophagitis, eosinophilic gastritis, eosinophilic gastroenteritis, eosinophilic colitis, eosinophilic jejunitis, eosinophilic duodenitis, eosinophilic pneumonia, eosinophilic fasciitis, eosinophilic cellulitis, eosinophilic vasculitis, eosinophilic myositis, allergies, asthma, atopic dermatitis, nasal polyposis, allergic rhinitis, drug eruption, drug hypersensitivity, eosinophilic cystitis, interstitial cystitis, bullous pemhigoid, bullous vegetans, primary immunodeficiency, acquired immunodeficiency syndrome (AIDS), infection such as invasive aspergillus fumigatus, allergic bronchopulmonary aspergillosis, eosinophilic leukemia, Churg-Strauss syndrome, and hypereosinophilic syndrome, and the like.

In some embodiments, the suppressor of EoP proliferation interacts with one or more TLRs. In some embodiments, the suppressor interacts with TLR4. In some embodiments, the suppressor interacts with a TLR2 heterodimer. In some embodiments, the suppressor interacts with TLR7.

In some embodiments, a suppressor of EoP proliferation that interacts with one or more TLRs is a TLR ligand. In other embodiments, the suppressor is a compound that interferes with a TLR so as to modulate TLR activity, without classical ligand-receptor binding character. In some embodiments, the suppressor is a compound that interferes with one or more TLRs so as to modulate TLR activity, without binding to the TLR. In some embodiments, the suppressor is a compound that triggers a TLR signal transduction pathway, without interfering with the TLR.

### TLR Ligands

Some embodiments of the invention relate to modulating one or more TLRs by administration of a ligand to one or more TLRs to suppress EoP proliferation, thereby treating an eosinophilia-associated condition. In some embodiments, TLRs that can be modulated include, for example, TLR4, TLR2 heterodimers (e.g. TLR2/TLR6 and TLR2/TLR1), or TLR7, or the like. In some embodiments, the ligand is a TLR4 ligand. In some embodiments, the ligand is a TLR2 heterodimer ligand. In some embodiments, the ligand is a TLR7 ligand.

Known ligands for TLRs include, for example, microbial ligands, synthetic ligands, and endogenous ligands, and the like (Kanzler, et al. Nat. Med. 13:552-9 (2007)). Known ligands for TLRs also include, for example, external molecules on bacterial, fungal, and protozoan pathogens, and the like.

Embodiments of the invention include the administration of known TLR ligands in order to treat an eosinophilic condition. In some embodiments, TLR ligands that can be used in the treatment of eosinophila-associated conditions include for example, but are not limited to, known TLR4 ligands, such as a lipopolysaccharide (LPS), mannan, phospholipids, envelope proteins such as MMTV and RSV, monophosphoryl lipid A (MPLA), ultrapure MPLA, synthetic MPLA (sMPLA), HSP-22, HSP-60, HSP-70, HSP-96, fibrinogen (extra domain A), minimally modified low-density lipoprotein, surfactant protein A, and the like. While these exemplary compounds are known TLR4 ligands, these compounds can be used in other embodiments as ligands for other TLRs. In some embodiments, TLR ligands that can be used in the treatment of eosinophilia-associated conditions include for example, but are not limited to, known TLR2 ligands, such as triacyl lipopeptide (Pam3CSK4), diacyl lipopeptides (Pam2CSK4), lipoteichoic acid, zymosan, porins, MALP2, bacterial peptidoglycan, lipoarabinomannan, HSP-60, HSP-70, HSP-96, HMGB1, and the like. While these exemplary compounds are known TLR2 ligands, these compounds can be used in other embodiments as ligands for other TLRs. In some embodiments, TLR ligands that can be used in the treatment of eosinophilia-associated conditions include for example, but are not limited to, known TLR7 ligands, such as ssRNA (viral), GU-rich oligoribonucleotides, loxoribin, CL264, imiquimod, resiquimod, adenosine and guinosine derivatives, ssRNA (immune complexes), and the like. While these exemplary compounds are known TLR7 ligands, these compounds can be used in other embodiments as ligands for other TLRs.

In some embodiments, the TLR ligand is an LPS. In some embodiments, the TLR ligand is a lipopeptide.

In some embodiments, TLR ligands that can be used in the treatment of eosinophilia-associated conditions include molecules that are structurally similar to those listed above. Structurally similar compounds are those that are not structurally identical but can have similar TLR inhibitory function, though the TLR inhibitory function can be substantially increased or decreased. Heretofore unknown TLR ligands can be contemplated and designed based on knowledge of a known TLR ligand.

In some embodiments, TLR ligands that can be used in the treatment of eosinophilia-associated conditions include, for example, lipids, lipopeptides, proteins, small molecules, and nucleic acids, and the like. TLR inhibitors for the treatment of eosinophilia-associated conditions can be identified by known methodologies.

The lists of specific TLR ligands presented herein are representative and not exhaustive. One of skill in the art can recognize other TLR ligands that can be used in the present invention.

Heretofore unknown TLR ligands can be developed by the screening of various compounds. Compounds that can be screened to determine their utility as TLR inhibitors include for example, but are not limited to, libraries of known compounds, including natural products, such as plant or animal extracts, synthetic chemicals, biologically active materials including proteins, peptides such as soluble peptides, including but not limited to members of random peptide libraries and combinatorial chemistry derived molecular libraries made of D- or L-configuration amino acids, or both, phosphopeptides (including, but not limited to, members of random or partially degenerate, directed phosphopeptide libraries), antibodies (including, but not limited to, polyclonal, monoclonal, chimeric, human, anti-idiotypic or single chain antibodies, and Fab, F(ab′)₂ and Fab expression library fragments, and epitope-binding fragments thereof), organic and inorganic molecules, and the like.

In addition to the more traditional sources of test compounds, computer modeling and searching technologies permit the rational selection of test compounds by utilizing structural information from the ligand binding sites relevant proteins. Such rational selection of test compounds can decrease the number of test compounds that must be screened in order to identify a therapeutic compound. Knowledge of the sequences of relevant proteins allows for the generation of models of their binding sites that can be used to screen for potential ligands. This process can be accomplished in several manners known in the art. A preferred approach involves generating a sequence alignment of the protein sequence to a template (derived from the crystal structures or NMR-based model of a similar protein(s), conversion of the amino acid structures and refining the model by molecular mechanics and visual examination. If a strong sequence alignment cannot be obtained then a model can also be generated by building models of the hydrophobic helices. Mutational data that point towards residue-residue contacts can also be used to position the helices relative to each other so that these contacts are achieved. During this process, docking of the known ligands into the binding site cavity within the helices can also be used to help position the helices by developing interactions that would stabilize the binding of the ligand. The model can be completed by refinement using molecular mechanics and loop building using standard homology modeling techniques. (General information regarding modeling can be found in Schoneberg, T. et. al. Molecular and Cellular Endocrinology 151:181-93 (1999); Flower, D. Biochimica et Biophysica Acta 1422:207-34 (1999); and Sexton, P. Current Opinion in Drug Discovery and Development 2:440-8 (1999).)

Once the model is completed, it can be used in conjunction with one of several existing computer programs to narrow the number of compounds to be screened by the screening methods of the present invention, like the DOCK program (UCSF Molecular Design Institute, San Francisco, Calif.). In several of its variants it can screen databases of commercial and/or proprietary compounds for steric fit and rough electrostatic complementarity to the binding site. Another program that can be used is FLEXX (Tripos Inc., St. Louis, Mo.).

### Administration

Administration of TLR-4, TLR-2, or TLR-7 ligands to suppress EoP prolifereation as disclosed herein can be used in methods of treating or preventing an eosinophilia-associated condition in a subject in need thereof.

TLR ligands can be administered by any pharmaceutically acceptable carrier, including, for example, any and all solvents, dispersion media, coatings, antibacterial and antifungal agents, isotonic and absorption delaying agents, and the like, compatible with pharmaceutical administration. The use of such media and agents for pharmaceutically active substances is known in the art. Except insofar as any conventional medium or agent is incompatible with the active compound, such media can be used in the compositions of the invention. Supplementary active compounds can also be incorporated into the compositions. A pharmaceutical composition of the invention is formulated to be compatible with its intended route of administration. Routes of administration include for example, but are not limited to, intravenous, intramuscular, and oral, and the like. Additional routes of administration include, for example, sublingual, buccal, parenteral (including, for example, subcutaneous, intramuscular, intraarterial, intradermal, intraperitoneal, intracisternal, intravesical, intrathecal, or intravenous), transdermal, oral, transmucosal, and rectal administration, and the like.

Solutions or suspensions used for appropriate routes of administration, including, for example, but not limited to parenteral, intradermal, or subcutaneous application, and the like, can include, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates, or phosphates and agents for the adjustment of tonicity such as sodium chloride or dextrose, and the like. The pH can be adjusted with acids or bases, such as, for example, hydrochloric acid or sodium hydroxide, and the like. The parenteral preparation can be enclosed in, for example, ampules, disposable syringes, or multiple dose vials made of glass or plastic, and the like.

Pharmaceutical compositions suitable for injectable use include, for example, sterile aqueous solutions (where water soluble) or dispersions and sterile powders for the extemporaneous preparation of sterile injectable solutions or dispersion, and the like. For intravenous administration, suitable carriers include, for example, physiological saline, bacteriostatic water, Cremophor ELTM (BASF, Parsippany, N.J.) or phosphate buffered saline (PBS), and the like. In all cases, the composition should be fluid to the extent that easy syringability exists. The carrier can be a solvent or dispersion medium containing, for example, water, ethanol, polyol (for example, glycerol, propylene glycol, and liquid polyethylene glycol, and the like), and suitable mixtures thereof, and the like. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, such as, for example, parabens, chlorobutanol, phenol, ascorbic acid, thimerosal, and the like. In many cases, it can be preferable to include isotonic agents, such as, for example, sugars, polyalcohols such as mannitol, sorbitol, and sodium chloride, and the like, in the composition. Prolonged absorption of the injectable compositions can be brought about by including in the composition an agent which delays absorption such as, for example, aluminum monostearate and gelatin, and the like.

Sterile injectable solutions can be prepared by incorporating the active compound (e.g., LPS) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets, for example. For oral administration, the agent can be contained in enteric forms to survive the stomach or further coated or mixed to be released in a particular region of the gastrointestinal (GI) tract by known methods. For the purpose of oral therapeutic administration, the active compound can be incorporated with excipients and used in the form of tablets, troches, or capsules, or the like. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches, and the like can contain any of the following exemplary ingredients, or compounds of a similar nature: a binder such as microcrystalline cellulose, gum tragacanth or gelatin; an excipient such as starch or lactose, a disintegrating agent such as alginic acid, Primogel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring, or the like.

For administration by inhalation, the compounds can be delivered in the form of an aerosol spray from pressured container or dispenser, which contains a suitable propellant, e.g., a gas such as carbon dioxide, or a nebulizer, or the like.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives, and the like. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems, and the like. Biodegradable, biocompatible polymers can be used, such as, for example, ethylene vinyl acetate, polyanhydrides, polyglycolic acid, collagen, polyorthoesters, and polylactic acid, and the like. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, which is incorporated herein by reference in its entirety.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The details for the dosage unit forms of the invention are dictated by and directly dependent on the unique characteristics of the active compound and the particular therapeutic effect to be achieved, and the limitations inherent in the art of compounding such an active compound for the treatment of individuals. Such details are known to those of skill in the art.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Production of Mature Eosinophils Via an Ex Vivo Liquid Culture System

An ex vivo culture system of whole bone marrow that results in functionally competent eosinophils was recently described (Dyer, et al. J. Immunol. 181:4004-9 (2008)). To investigate molecular regulators of eosinophil differentiation, the culture system was adapted to start with low-density bone marrow (LDBM) cells.

### Mice

In-house bred, four- to six-week-old, male and female wild-type BALB/c mice were used as a source of bone marrow cells unless otherwise indicated. Wild-type C57BL/6 (CD45.1) and TLR4-deficient mice (C57BL/6, CD45.2) (The Jackson Laboratory, Bar Harbor, Me.) were allowed to acclimatize for at least 2 weeks prior to use. All mice were housed under specific pathogen-free conditions and treated according to institutional guidelines.

### Murine Eosinophil Liquid Cultures

The femurs and tibiae of mice were crushed in a sterile mortar and pestle containing 1×PBS with 2% FBS, and cells were collected by filtration through a 70-μm strainer. Red blood cells were lysed using RBC lysis buffer (Sigma, St. Louis, Mo.), and the remaining cells were subjected to centrifugation at 1700 rpm for 30 minutes on a Histopaque 1083 (Sigma) gradient. LDBM cells were aspirated from the interface, washed twice with 1× PBS and cultured at 0.8-1×106 cells per ml in 6-well plates or 24-well plates containing Complete Medium composed of Iscove's Modified Dulbecco's Medium (IMDM) with Glutamax (Gibco, Carlsbad, Calif.), 10% characterized FBS (HyClone, Hudson, N.H.), 2 mM L-glutamine (Gibco), 50 μM 2-ME (Sigma), and penicillin and streptomycin (Gibco) supplemented with 100 ng/mL stem cell factor (SCF; Peprotech, Rocky Hill, N.J.) and 100 ng/mL FLT3-ligand (FLT3L; Peprotech). On day 4, the medium in each non-centrifuged plate was replaced with fresh Complete Medium containing 10 ng/mL murine IL-5 (Peprotech). Medium with IL-5 was changed every 2 days thereafter. On Day 8, some cultures were treated with LPS from E. coli serotype O55:B5 (Sigma), ultrapure LPS from S. minnesota (InvivoGen, San Diego, Calif.), monophosphoryl lipid A from S. minnesota R595LPS (InvivoGen), synthetic lipid A from E. coli (InvivoGen), Pam2CSK4 (InvivoGen), Pam3CSK4 (InvivoGen), imiquimod (InvivoGen), CL264 (InvivoGen), or poly(I:C) (InvivoGen) at indicated doses in the presence of IL-5.

After TLR ligand exposure for 18-24 hours, the medium was changed to Complete Medium with IL-5 at 10 ng/ml for the remaining culture period. For neutralization experiments, monoclonal anti-mouse IFN-γ (clone 37895; R&D Systems, Minneapolis, Minn.) or rat IgG2a isotype antibody was added at 0.3 μg/ml to media during LPS exposure and to Complete Medium with IL-5 thereafter. For some experiments, cytokines (IL-6 at 0.1 ng/ml, IL-10 at 2 ng/ml, IFN-γ at 0.5 ng/ml, TNF-α at 0.5 ng/ml, or the combination of all four) were added on Day 8 to IL-5-containing Complete Medium. Cells were cultured in the presence of these individual cytokines or the cytokine combination for the remainder of the culture period.

### Surface Marker and Receptor Expression

Whole bone marrow or LDBM cells were washed twice in 1×PBS containing 1% FBS and 1 mM EDTA at a concentration of 4×106 cells/mL and blocked with 0.5 μg rat anti-mouse CD16/CD32 (BD Bioscience, San Diego, Calif.) for 5 minutes at 4° C. Cells were incubated with fluorescent-labeled antibodies for 30 minutes at 4° C. and then washed twice and resuspended in flow cytometry buffer. Antibodies used include rat anti-mouse CD34-FITC (BD Bioscience), rat anti-mouse TLR4-PE (Imgenex, San Diego, Calif.), rat anti-mouse Siglec-F-PE (BD Bioscience), and rat anti-mouse CCR3-FITC (R&D Systems). All flow cytometric data were acquired using a FACS Calibur or FACS Canto flow cytometer (BD Bioscience).

### Results

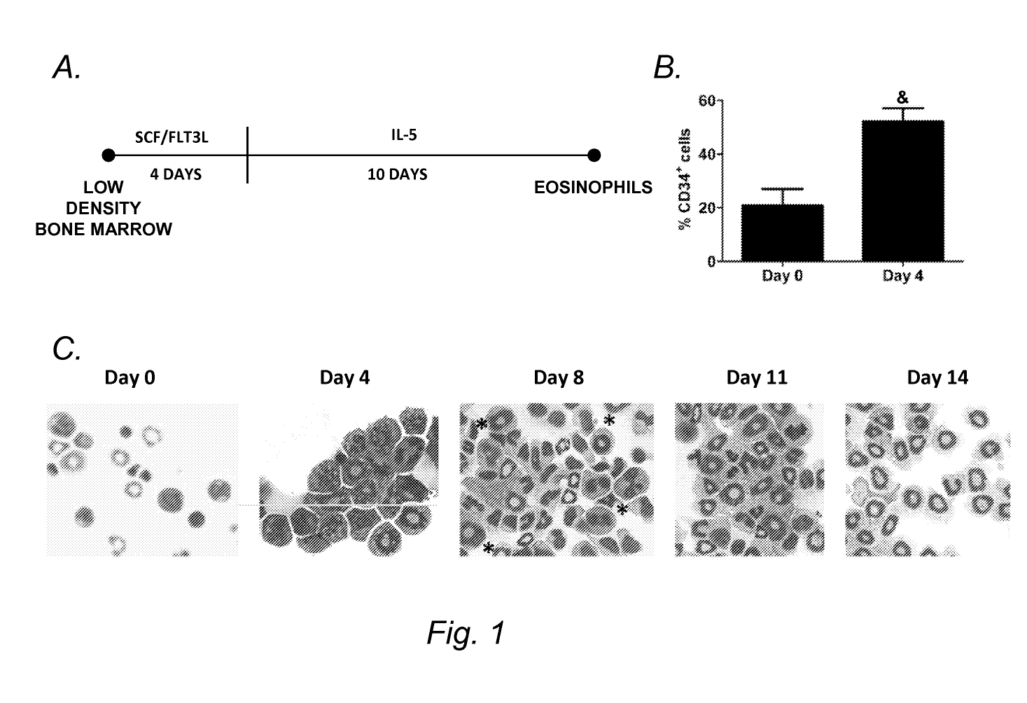

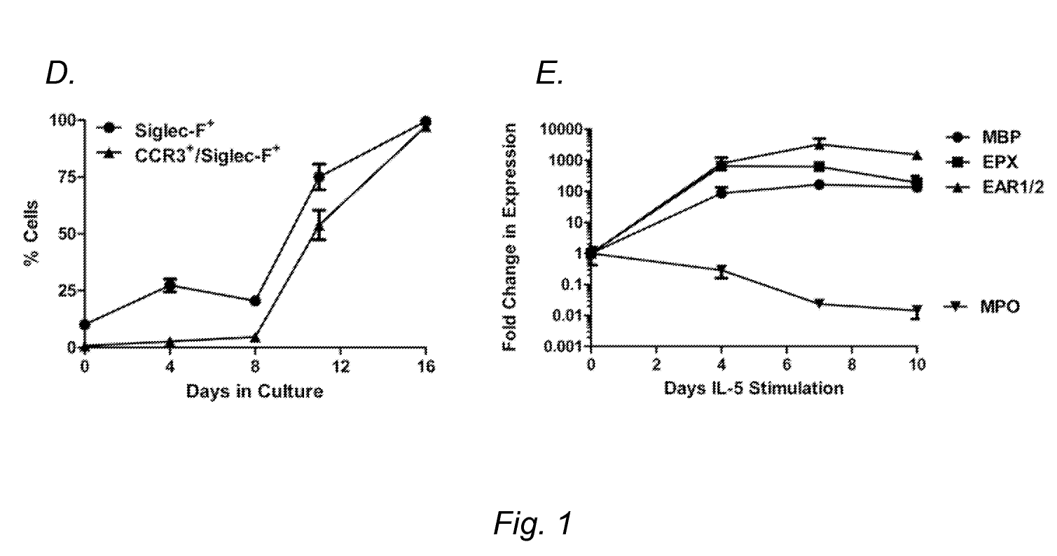

The LDBM population was enriched for CD34⁺ progenitors to reduce any influence of more mature cells on the cultures (FIG. 1A). Stimulation of LDBM cells with SCF and FLT3L for 4 days resulted in a 2.5-fold increase in CD34′ progenitors (FIG. 1B). The CD34⁺-enriched cells were subsequently exposed to IL-5 (FIG. 1A). Cell morphology was monitored from day 0 to day 14, revealing evidence of granularity within 4 days of IL-5 stimulation (FIG. 1C).

Expression of surface markers associated with eosinophils, namely Siglec-F and CCR3, was detected after 7 days of IL-5 stimulation, with greater than 98% of the cultured cells being positive for both Siglec-F and CCR3 after 14 days in culture (FIG. 1D). Expression of the mRNA for granule proteins dramatically increased within 4 days of IL-5 stimulation, correlating with the development of granule morphology within the same time period (FIGS. 1C and 1E). In contrast, expression of the neutrophil-specific enzyme myeloperoxidase decreased throughout the culture period (FIG. 1E).

FIG. 1A. A schematic of the eosinophil liquid culture system is depicted. LDBM cells are cultured in media containing the cytokines SCF and FLT3L for 4 days to expand the progenitor population, after which the cells are cultured in media supplemented with IL-5 alone for an additional 10 days to yield mature eosinophils.

FIG. 1B. Percentage (mean SEM) of CD34-expressing cells in the cultures before (day 0) and after (day 4) stimulation with SCF and FLT3L is depicted, as determined by flow cytometry (n=3 samples per time point per experiment with 3 independent experiments). ^{&}P<0.0001 when compared to day 0 cells.

FIG. 1C. Representative cytospin preparations of LDBM cells harvested from cultures at the indicated time points are depicted. Magnification of 400×. Granule-containing cells (asterisks, *) are evident after 4 days of IL-5 exposure.

FIG. 1D. Percentage (mean SD, n=3-5 wells per time point) of LDBM cells expressing surface Siglec-F (circles) or CCR3 and Siglec-F (triangles) in the cultures at the indicated time points is depicted, as determined by flow cytometry (n=5 experiments with representative experiment shown).

FIG. 1E. Fold change (mean SEM) in normalized mRNA levels for major basic protein (MBP; circles), eosinophil peroxidase (EPX; squares), eosinophil ribonuclease 1 and 2 (EAR1/2; triangles), and myeloperoxidase (MPO; inverted triangles) in cultured LDBM cells are depicted at the indicated time points (n=3 samples per time point per experiment with 3 independent experiments).

### Example 2

### TLR Expression in Developing Eosinophils

Previous studies have demonstrated TLR4 expression on a percentage of by early hematopoietic progenitors and by mature eosinophils (Nagai, et al. Immunity 24:801-12 (2006)). Despite the challenge in detecting TLR4 on the surface of these rare cells, a response to a TLR4 ligand was noted with sorted HSCs and GMPs from which EoPs arise (Nagai, et al. Immunity 24:801-12 (2006)). Mature eosinophils have been shown to express the transcripts for a number of different TLRs, including TLR4 (Nagase, et al. J. Immunol. 171:3977-82 (2003); Plotz, et al. Blood 97:235-41 (2001); Sabroe, et al. J. Immunol. 168:4701-10 (2002)), although most mature eosinophils from wild-type mice and normal human donors have no detectable surface expression of TLR4 despite detection of mRNA expression (Nagase, et al. J. Immunol. 171:3977-82 (2003); Sabroe, et al. J. Immunol. 168:4701-10 (2002); Wong, et al. Am. J. Respir. Cell. Mol. Biol. 37:85-96 (2007); Driss, et al. Blood 113:3235-44 (2009); Phipps, et al. Blood 110:1578-86 (2007)).

Intracellular TLR4 protein has been detected in both murine and human mature eosinophils, although the significance of this intracellular reserve is unclear (Wong, et al. Am. J. Respir. Cell. Mol. Biol. 37:85-96 (2007); Driss, et al. Blood 113:3235-44 (2009); Phipps, et al. Blood 110:1578-86 (2007)). Most studies demonstrate a complete lack of response of purified mature eosinophils to LPS exposure (Nagase, et al. J. Immunol. 171:3977-82 (2003); Sabroe, et al. J. Immunol. 168:4701-10 (2002); Wong, et al. Am. J. Respir. Cell. Mol. Biol. 37:85-96 (2007)), supporting specificity for the EoP rather than the whole eosinophil lineage.

Expression of TLRs by EoPs and their functional response to TLR activation during eosinophil differentiation has heretofore not been investigated. Therefore, a study was designed to determine TLR expression during eosinophil differentiation.

### Gene Expression

Total cellular RNA was extracted with Trizol (Invitrogen, Carlsbad, Calif.), according to the manufacturer's instructions, and further purified with the Rneasy Mini Kit (Qiagen, Valencia, Calif.). Reverse transcription was performed using Iscript (Bio-Rad, Hercules, Calif.). Real-time PCR was performed by rapid-cycling using the IQ5 SYBR mix (Bio-Rad) as a ready-to-use reaction mix according to the manufacturer's instructions. Expression levels for each mRNA were normalized to expression of a housekeeping gene (GAPDH or TATA-binding protein) in the cDNA sample tested.

### Isolation of TLR4-Expressing Cells

Biotinylation of the azide-free TLR4 antibody (clone MTS510) (Imgenex) was performed using the One-Step Antibody Biotinylation Kit (Miltenyi Biotec, Auburn, Calif.). LDBM cells were cultured, as described above. On day 8, cells were washed and resuspended in ice-cold auto-MACS running buffer (Miltenyi Biotec) at a concentration of 2.5×108 cells/mL. All centrifugation was performed at 300×g, and cells were kept on ice at all times. Cells were magnetically labeled with 25-100 μg of biotinylated-TLR4 antibody and 20 μL of anti-biotin microbeads per 107 cells (Miltenyi Biotec). Cells were then washed and resuspended in autoMACS running buffer at a concentration of 108 cells/mL, then subjected to MACS separation using LS columns and a QuadroMACS™ separator (Miltenyi Biotec). Cells were then washed in pre-warmed Complete Medium with 10 ng/mL of IL-5 and either set aside for flow cytometric analysis or cultured in 24-well dishes for an additional 6 days with media changes every 48 hours.

Immediately following MACS separation, separated cells were stained with FITC-TLR4/MD2 (clone MTS510) (Invivogen) and PE-CD34 (BD Bioscience). Positively selected cells were greater than 92% TLR4. After 6 days in culture, cells were stained with FITC-CCR3 (R&D Systems), PE-Siglec-F (BD), and LIVE/DEAD near IR viability dye (Invitrogen).

### Results

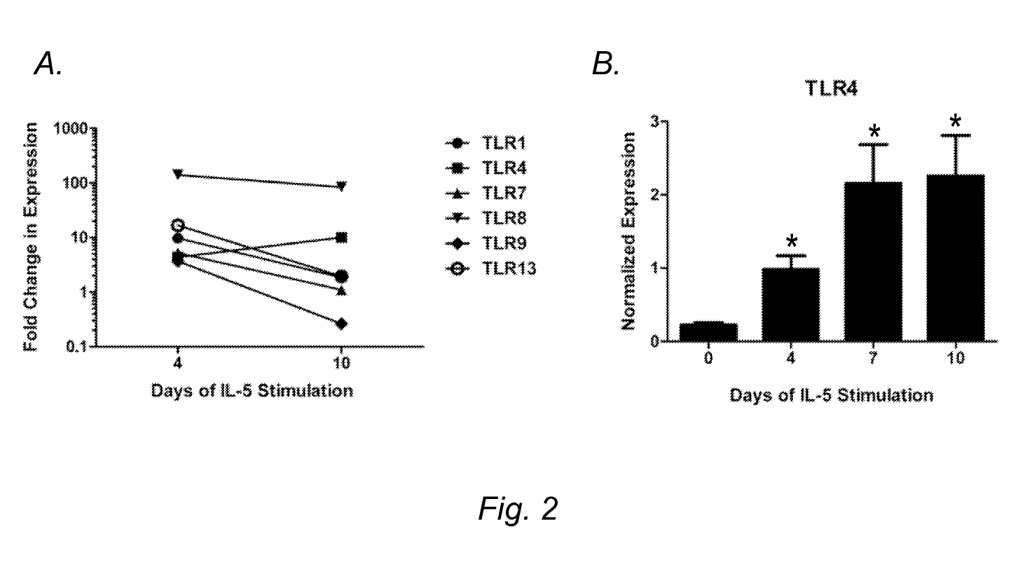

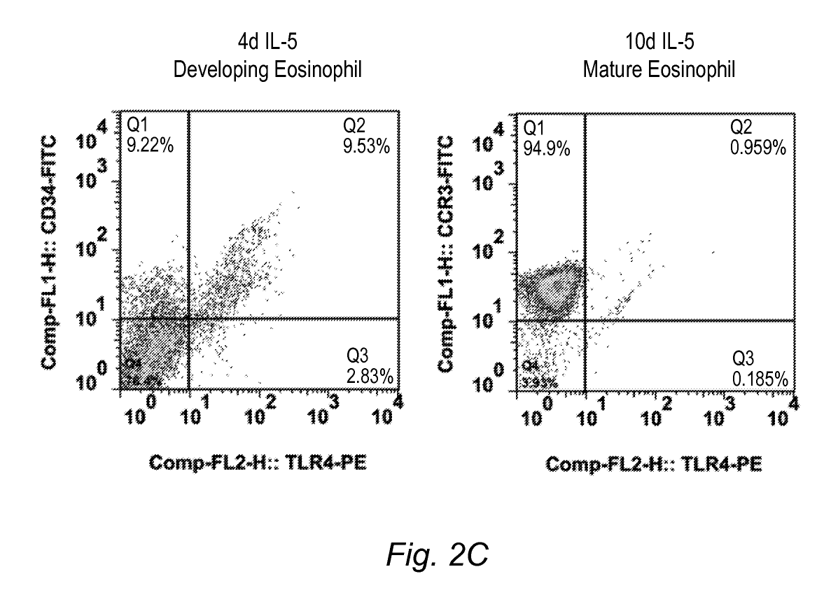

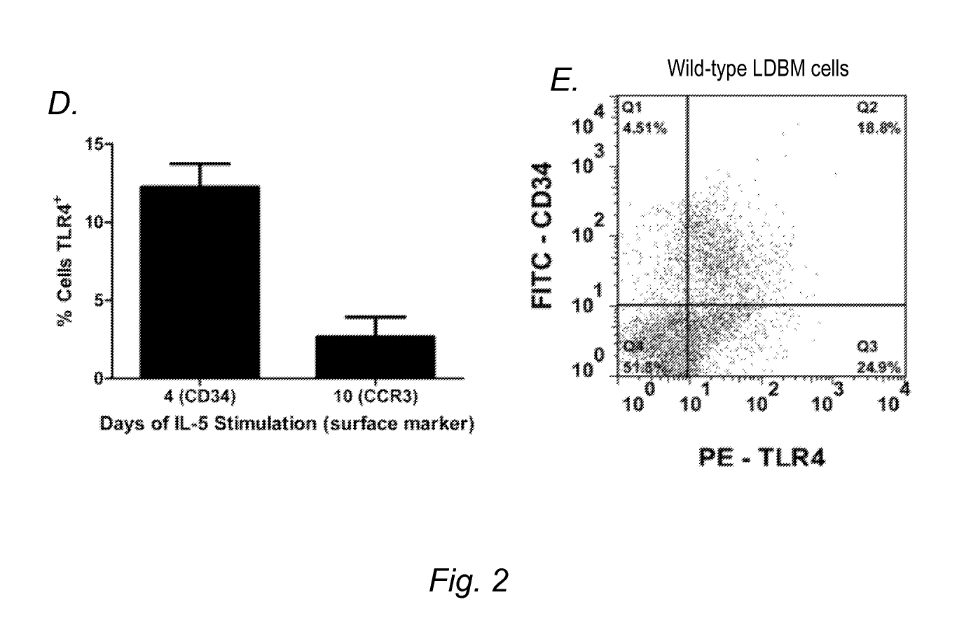

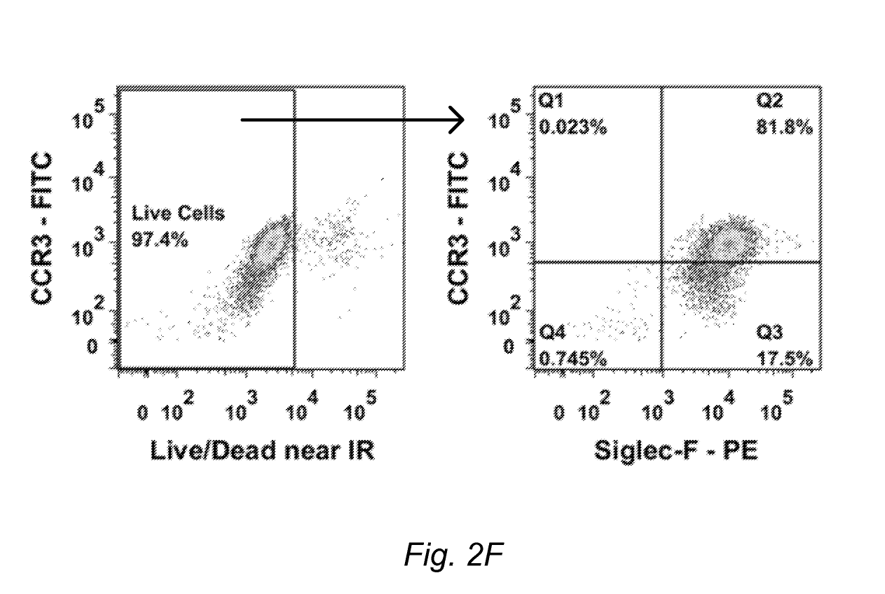

Expression of TLR1, TLR4, TLR7, TLRS, TLR9, and TLR13 mRNA was induced after 4 days of IL-5 stimulation of LDBM cells (FIG. 2A and Table 1). As TLR4 was one of the more highly expressed TLRs by the early EoP (prior to surface expression of Siglec-F and CCR3), the expression of TLR4 protein on EoPs was further investigated. Expression of TLR4 mRNA was induced after 4 days of IL-5 stimulation, peaked after 7 days, and remained elevated after 10 days of IL-5 stimulation (FIG. 2B).

In the culture system used, IL-5 stimulation of EoPs resulted in upregulated surface expression of TLR4 protein on developing eosinophils (FIG. 2C). Expression of TLR4 protein was subsequently down-regulated from the surface, as the EoPs matured after 10 days of IL-5 stimulation (FIGS. 2C and 2D). TLR4 protein expression was confirmed on the surface of CD34-expressing progenitors in the bone marrow of wild-type mice (FIG. 2E).

To determine whether the TLR4-expressing cells were EoPs, TLR4⁺ cells from Day 8 cultured cells were purified (after 4 days of IL-5 stimulation), then this TLR4⁺ population was cultured in IL-5 for another 6 days. Greater than 95% of the TLR4⁺ cells isolated at Day 8 expressed Siglec-F, and greater than 80% of these cells co-expressed the eosinophil markers CCR3 and Siglec-F at day 14, confirming that EoPs are TLR4⁺ (FIG. 2F). The findings of increased expression of TLR4 mRNA in response to IL-5 stimulation of murine bone marrow progenitors and of detected TLR4 on the surface of murine CD34⁺ precursors after 4 days of IL-5 stimulation are consistent with the previous studies described above.

FIG. 2A. Fold changes (mean SD) in mRNA levels in LDBM cells cultured in IL-5 are depicted for the indicated times as compared to mRNA levels before IL-5 stimulation (n=4-6 samples per time point).

FIG. 2B. Normalized expression (mean SD) of TLR4 mRNA in LDBM cells cultured in IL-5 is depicted for the indicated times (n=4-6 samples per time point). *P<0.05 when compared to cells prior to IL-5 stimulation.

FIG. 2C. Surface expression of TLR4 on developing eosinophils (CD34) or on mature eosinophils (CCR3) after 4 and 10 days of IL-5 stimulation, respectively, is shown by a representative density blot from 4 experiments.

FIG. 2D. The percentages of cultured cells co-expressing TLR4 with a marker for the progenitor population (CD34) or for a mature eosinophil (CCR3) after 4 or 10 days of IL-5 stimulation, respectively, are depicted (mean SEM, n=4 independent experiments).

FIG. 2E. Surface expression of CD34 and TLR4 on freshly isolated wild-type LDBM cells is shown by a representative density blot from 2 experiments.

FIG. 2F. Viability and surface expression of CCR3 and Siglec-F at Day 14 on TLR4⁺ cells that were isolated on Day 8 and cultured for another 6 days in IL-5. Representative density blots from 2 experiments are shown.

TABLE 1
Normalized TLR expression in developing eosinophils

| TLR | Day 0 | Day 4 | Day 7 | Day 10 |
|---|---|---|---|---|
| TLR1 | 0.029 (0.011) | 0.283 (0.089) | 0.195 (0.115) | 0.054 (0.028) |
| TLR2 | 0.423 (0.187) | 1.301 (0.772) | 2.195 (1.323) | 0.587 (0.346) |
| TLR4 | 0.224 (0.057) | 0.983 (0.417) | 2.155 (1.296) | 2.257 (1.353) |
| TLR6 | 0.164 (0.207) | 0.058 (0.039) | 0.087 (0.064) | 1.903 (3.831) |
| TLR7 | 0.149 (0.094) | 0.780 (0.299) | 0.464 (0.186) | 0.164 (0.031) |
| TLR8 | 0.0008 (0.0009) | 0.1128 (0.1013) | 0.0137 (0.0160) | 0.0674 (0.0467) |
| TLR9 | 0.099 (0.042) | 0.367 (0.117) | 0.102 (0.057) | 0.026 (0.007) |
| TLR13 | 0.036 (0.015) | 0.603 (0.331) | 0.939 (0.372) | 0.070 (0.162) |

Transcript levels as determined by quantitative PCR and normalized to the housekeeping gene TATA-binding protein. Data presented as mean (SD) of 3-6 samples

### Example 3

### Inhibition of IL-5-Induced Eosinophil Production by TLR4 Activation Via LPS or LPS Mimetic

EoPs were found to express TLR4 (Example 2). Therefore, a subsequent study was designed to explore the functional response of EoPs to TLR4 activation.

### Results

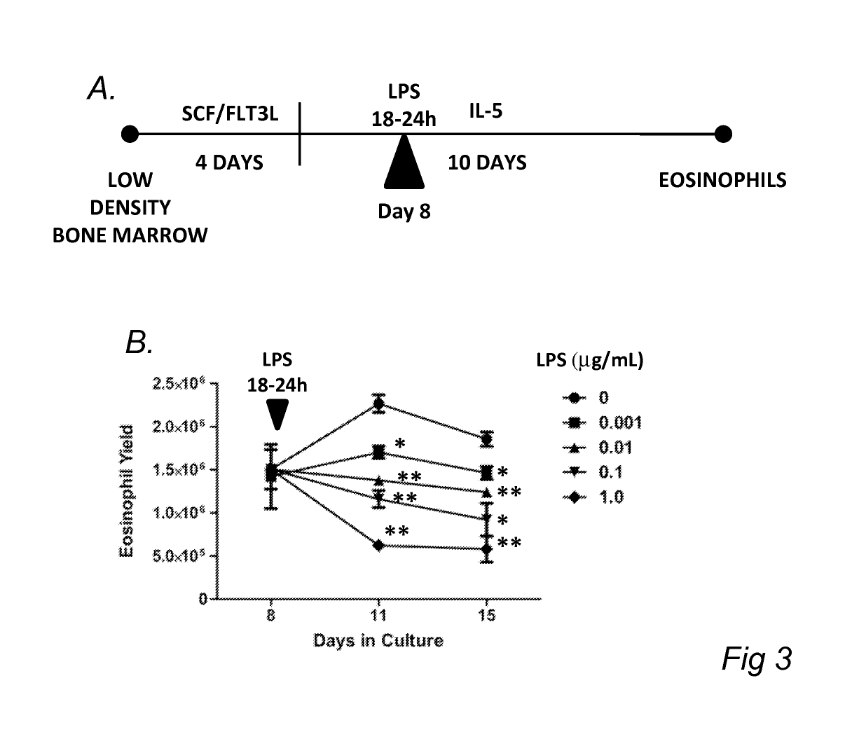

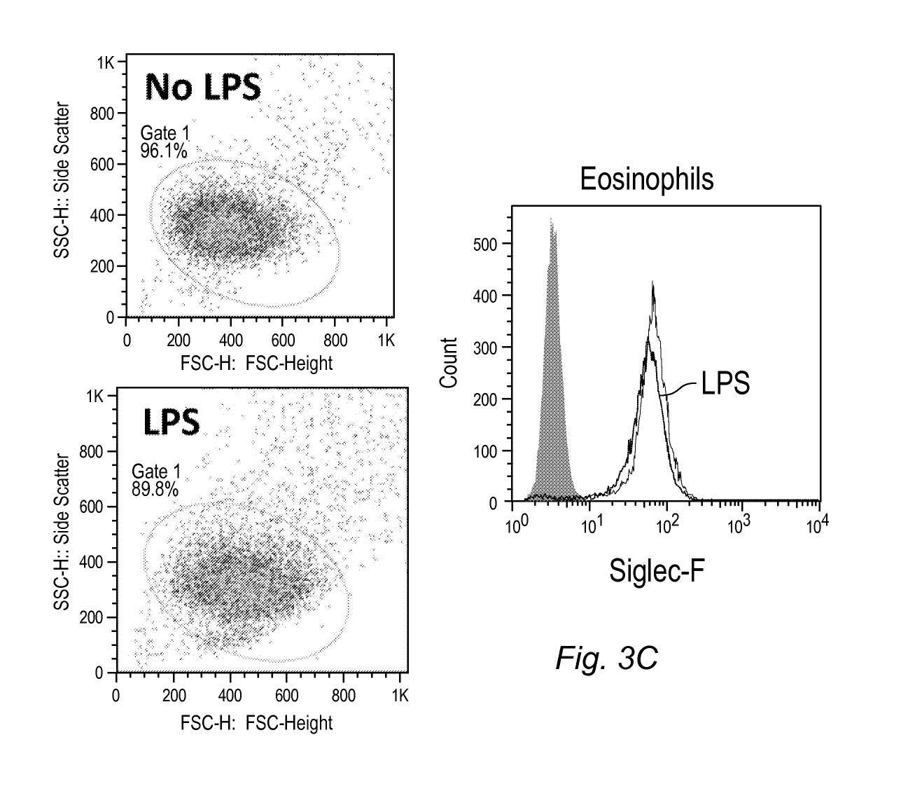

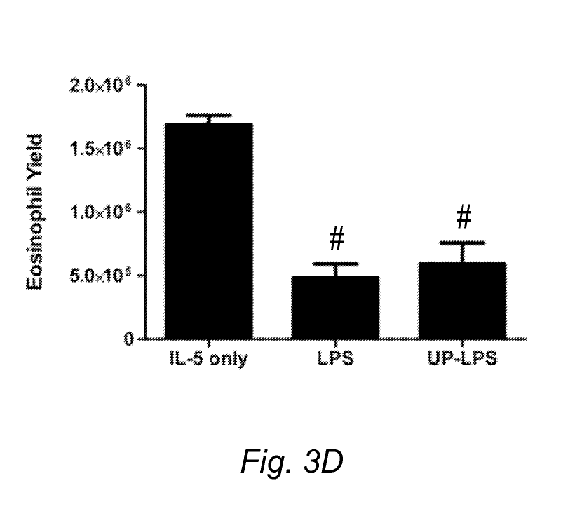

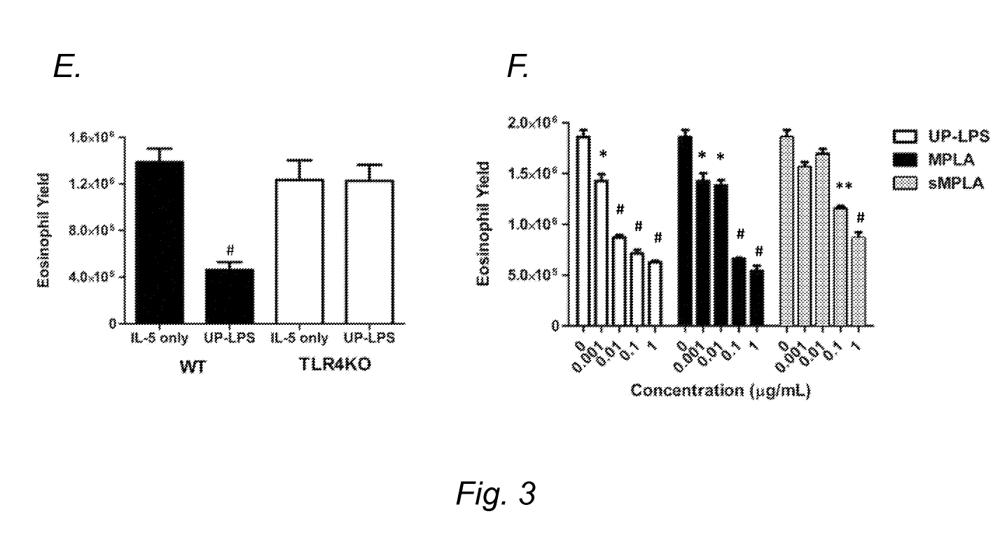

Developing eosinophils were exposed to the TLR4 ligand LPS for 18-24 hours after 4 days of IL-5 stimulation and subsequently continued with IL-5 stimulation alone for the remaining 6-7 days of culture (FIG. 3A). Treatment of developing eosinophils with LPS resulted in a dose-dependent reduction in total eosinophil yield in the culture (FIG. 3B). Although LPS exposure significantly reduced the eosinophil yield, IL-5-mediated differentiation of developing eosinophils was unaffected, as evidenced by the unchanged percentage of cells expressing Siglec-F (FIG. 3C).

As LPS preparations can contain bacterial products that stimulate multiple TLR signaling pathways, developing eosinophils were treated with an ultrapure preparation of LPS that only activates TLR4. Similar results were obtained with LPS and ultrapure LPS, substantiating that the suppression in eosinophil production is mediated primarily via TLR4 (FIG. 3D). Further, yield of TLR4-deficient eosinophils was unaffected by treatment with ultrapure LPS, confirming that expression of TLR4 is necessary for LPS-mediated suppression of eosinophil production (FIG. 3E).

Developing eosinophils were then treated with monophosphoryl lipid A (MPLA), a derivative of lipid A that exhibits adjuvant properties and activates TLR4 and TLR2 (Martin, et al. Infect. Immun. 71:2498-507 (2003)). Treatment of EoPs with MPLA resulted in a dose-dependent suppression of eosinophil production that was similar at the higher doses to that observed with LPS (FIG. 3F). Further, stimulation of developing eosinophils with a synthetic MPLA (sMPLA) that is structurally similar to natural MPLA but activates only TLR4 also resulted in significant suppression of eosinophil production at higher doses (FIG. 3F).

FIG. 3A. A schematic of LPS exposure for eosinophil progenitors is depicted. LDBM cells were stimulated with SCF and FLT3L for 4 days and subsequently cultured with IL-5 for 4 days to initiate eosinophil differentiation. On Day 8, cultured cells were stimulated with LPS and IL-5 for 18-24 hours, after which the medium was changed, and the cells were cultured for another 6 days with IL-5 alone.

FIG. 3B. Eosinophil yields at day 11 and day 15 with and without LPS stimulation at the indicated doses are depicted (mean SEM, n=3 independent experiments with 5-6 wells per time point per experiment). *P<0.05 and **P<0.01 when compared to yield with no LPS exposure at that time point.

FIG. 3C. Representative forward and side scatter plots of day 15 cultured cells and a histogram of Siglec-F surface expression of gated cells with and without LPS stimulation are shown (n=3 independent experiments).

FIG. 3D. Eosinophil yield at day 15 with and without LPS or ultrapure (UP-) LPS stimulation is shown (mean SEM, n=3 independent experiments with 5-6 wells per time point per experiment). ^{#}P<0.001 when compared to yield with IL-5 only.

FIG. 3E. Wild-type (WT, solid bars) and TLR4-deficient (TLR4KO, open bars) eosinophil yields at day 14 with and without UP-LPS exposure are depicted (mean SD, representative experiment of 3 experiments is shown, with 5-6 wells per time point). ^{#}P<0.001 when compared to yield with IL-5 only.

FIG. 3F. Eosinophil yields at day 14 following UP-LPS (open bars), derived monophosphoryl lipid A (MPLA, black bars), or synthetic monophosphoryl lipid A (sMPLA, shaded bars) exposure at the indicated doses are shown (mean SEM, n=2 independent experiments, with 6 wells per time point per experiment). *P<0.05, **P<0.01, and ^{#}P<0.001 when compared to yield from cultures with IL-5 only.

### Example 4

### Inhibition of Eosinophil Production Via Activation of TLR2 and TLR7 but not TLR3

TLR4 ligands were demonstrated to suppress eosinophil production (Example 3). Therefore, a study was designed to determine if suppression of eosinophil production was limited to TLR4 ligands.

### Results

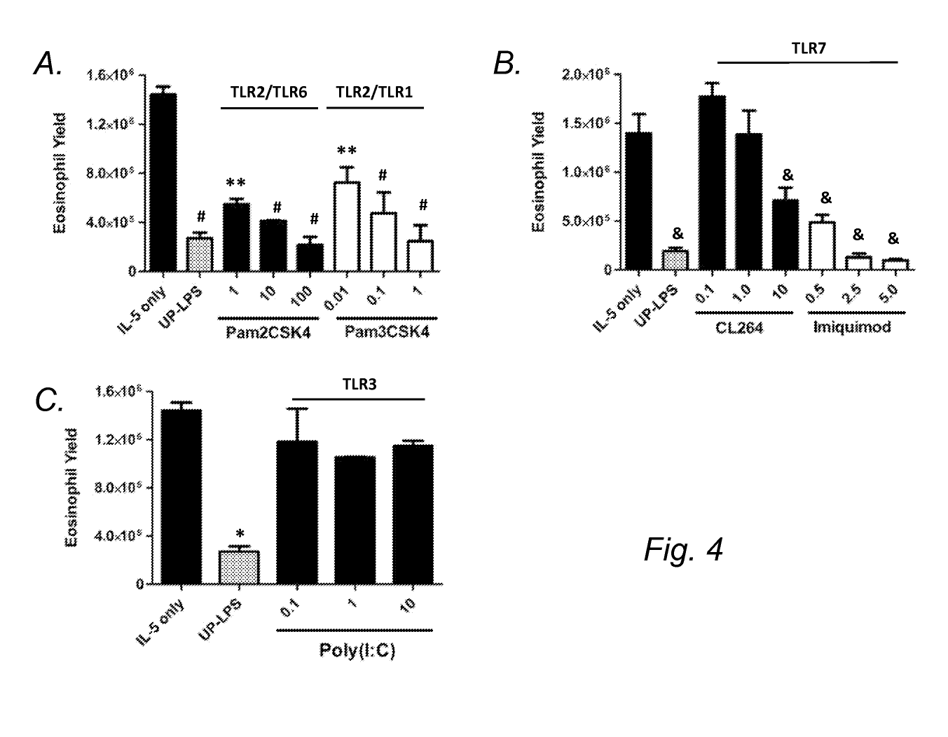

Developing eosinophils were exposed to synthetic lipopeptides (Pam2CSK4 or Pam3CSK4) that signal through the heterodimers TLR2 and TLR6 (Pam2CSK4) or TLR2 and TLR1 (Pam3CSK4). Treatment of cultures with Pam2CSK4 or Pam3CSK4 resulted in a dose-dependent reduction in eosinophil yield similar to that seen with ultrapure LPS (FIG. 4A). Exposure of developing eosinophils to adenine and guanosine analogs that specifically activate TLR7, CL264, and imiquimod, respectively, resulted in significant suppression of eosinophil yield (FIG. 4B). In contrast, the TLR3 ligand poly(I:C) had no effect on eosinophil production (FIG. 4C). Collectively, these results demonstrate specific inhibition of eosinophil production by ligands to TLR2 heterodimers, TLR4, and TLR7, but not TLR3.

FIG. 4A. Eosinophil yield at day 14 following exposure to TLR2 ligands (G, Pam2CSK4 and Pam3CSK4) or UP-LPS at the indicated doses is depicted (mean SEM, n=2 independent experiments, with 6 wells per time point per experiment). **P<0.01 and ^{#}P<0.001 when compared to yield with IL-5 only.

FIG. 4B. Eosinophil yield at day 14 following exposure to TLR7 ligands (H, CL264 and Imiquimod) or UP-LPS at the indicated doses is depicted (mean SEM, n=2 independent experiments, with 6 wells per time point per experiment). ^{&}P<0.0001 when compared to yield with IL-5 only.

FIG. 4C. Eosinophil yield at day 14 following exposure to TLR3 ligand (I, Poly(I:C)) or UP-LPS at the indicated doses is depicted (mean SEM, n=2 independent experiments, with 6 wells per time point per experiment). *P<0.05 when compared to yield with IL-5 only.

### Example 5

### Production of Pro-Inflammatory Cytokines Via TLR4 Activation

The TLR4 ligand LPS was demonstrated to suppress eosinophil production (Example 3). Therefore, a study was designed to determine whether LPS stimulation of EoPs would result in accumulation of pro-inflammatory mediators.

### Cytokine Quantitation

Cells and culture media were collected from 24-well plates at indicated time points in the culture period and centrifuged to pellet cells. Supernatants were transferred to clean tubes and frozen at −80° C. until thawed for cytokine quantitation. Protein levels of cytokines were quantified by using ELISA kits specific for IL-6 (R&D Systems), IL-10 (R&D Systems), IFN-γ (R&D Systems), and TNF-α (BioLegend, San Diego, Calif.) according to the manufacturer's instructions. Each condition was represented by 5-6 individual wells at each time point.

### Results

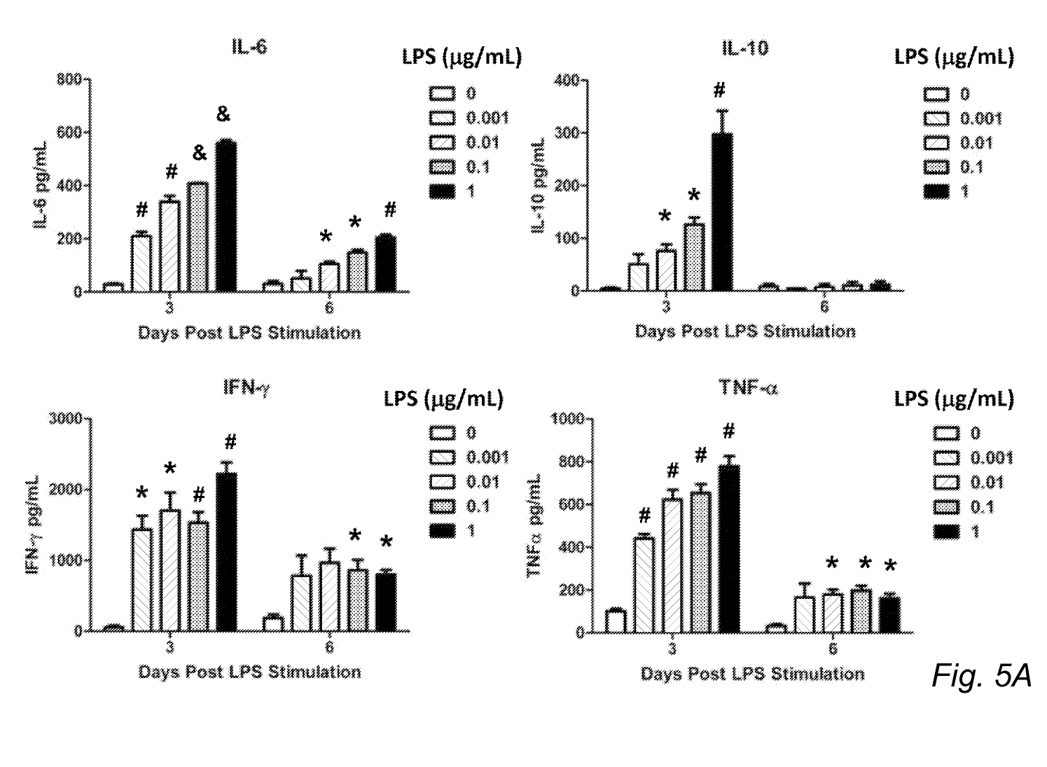

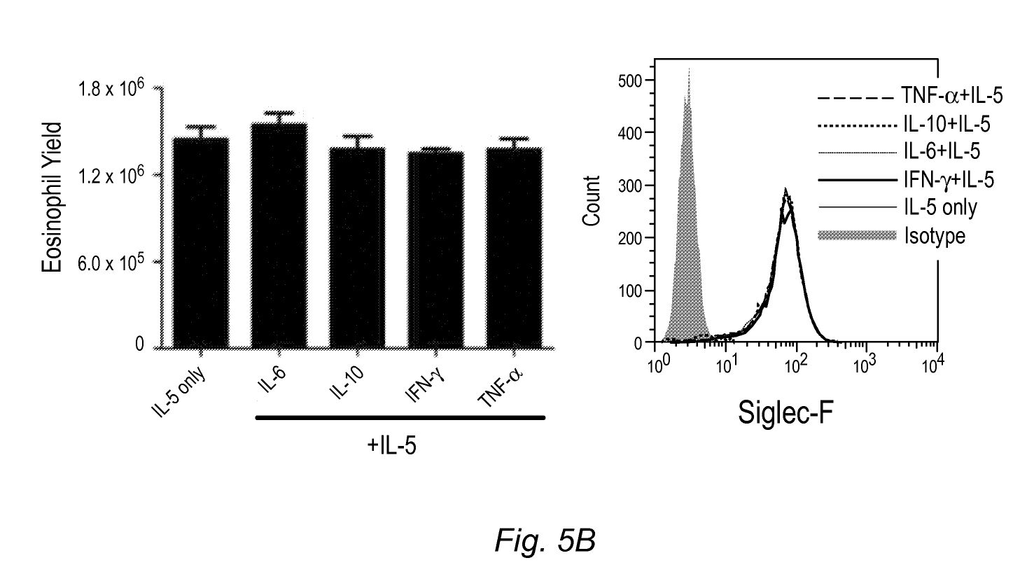

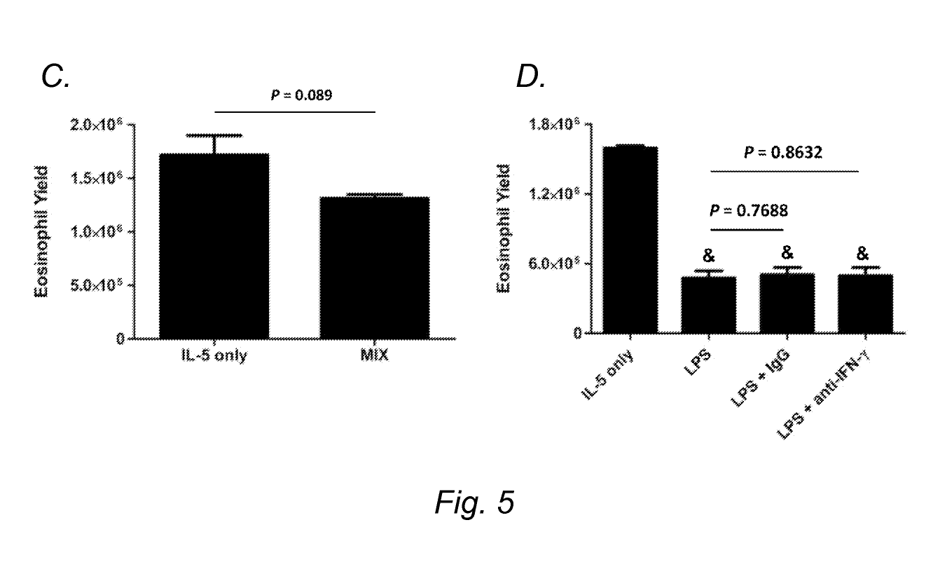

LPS stimulation increased production of IL-6, IL-10, IFN-γ, and TNF-α in a dose-dependent manner 3 days following LPS stimulation of EoPs, and mediator levels remained modestly elevated at 6 days after LPS exposure (FIG. 5A). To determine if these pro-inflammatory cytokines contribute to the LPS-induced suppression of eosinophil production, IL-5-stimulated EoPs were treated with IL-6, IL-10, IFN-γ, or TNF-α for the final 6 days of culture, and the effects on eosinophil differentiation and yield were measured.

Treatment of EoPs with IL-5 and IL-6, IL-10, IFN-γ, or TNF-α had no significant effect on the number of mature eosinophils produced (FIG. 5B). To determine if the LPS-induced pro-inflammatory mediators work synergistically to suppress eosinophil production, developing eosinophils were stimulated with the cytokine cocktail (L-5, IL-6, IL-10, IFN-γ, and TNF-α). Exposure to the cytokine cocktail did not result in a significant or consistent reduction in yield of mature eosinophils in the culture system used (FIG. 5C). Together, these studies demonstrate that EoPs have increased production of potent inflammatory mediators, such as TNF-α, following LPS exposure and that these same mediators have no major effect on eosinophil-lineage maturation or expansion.

As IFN-γ has been shown to inhibit eosinophil differentiation (de Bruin, et al. Blood 116:2559-69 (2010)), the role of LPS-induced IFN-γ on EoP differentiation was further investigated. LPS exposure was found to result in a similar reduction in the number of mature eosinophils produced irrespective of the presence or absence of neutralizing antibodies for IFN-γ, suggesting that LPS suppression of eosinophil differentiation is independent of IFN-γ production (FIG. 5D).

FIG. 5A. Concentrations of IL-6, IL-10, IFN-γ, and TNF-α in culture supernatants 3 and 6 days after LPS stimulation at indicated doses are depicted (mean SEM, n=3 experiments, with 5-6 samples per time point in each experiment). *P<0.05, ^{#}P<0.001, and ^{&}P<0.0001 when compared to cytokine concentration in supernatant from cultures with IL-5 only.

FIG. 5B. Eosinophil yields at Day 14 from cultures with IL-Sonly or with IL-5 and the indicated cytokines are shown (mean SEM, n=4 experiments, with 6 samples from each condition per experiment). A representative histogram indicating surface Siglec-F expression on day 14 from cells cultured with IL-5 only or with IL-Sand IFN-γ, IL-6, IL-10, or TNF-α for the final 6 days of the culture is shown (n=3 experiments). Isotype control antibody staining is represented by the filled gray line.

FIG. 5C. Eosinophil yields at day 15 from cultures with IL-5 only or with IL-5 with a mixture (MIX) of cytokines (IL-5, IL-6, IL-10, IFN-γ, and TNF-α) for the final 6 days of the culture are shown (mean SEM, n=3 experiments, with 6 samples from each condition per experiment).

FIG. 5D. Eosinophil yields at day 15 from cultures without LPS exposure (IL-5 only), exposed to LPS, or exposed to LPS with a neutralizing anti-IFN-γ antibody or its isotype control antibody (IgG, n=3 experiments, with 6 samples from each condition per experiment) are shown. ^{&}P<0.0001 when compared to cultures without LPS exposure.

### Example 6

### Reduction in EoPs Proliferation but not Viability Via LPS Exposure

In sepsis, apoptosis of lymphocytes markedly increases in the thymus and bone marrow in both mice and humans (Hotchkiss et al. J. Immunol. 166:6952-63 (2001); Wang, et al. J. Immunol. 152:5014-21 (1994); Ayala, et al. Blood 87:4261-75 (1996)). Therefore, the effect of LPS on IL-5-induced expansion of EoPs with pulse-chase experiments was investigated, and EoP viability was examined following LPS exposure to determine if LPS-induced suppression of eosinophil production is mediated via induction of apoptosis of developing eosinophils.

### Proliferation Detection

LDBM cells were cultured and treated with LPS, as described previously. Cells were the pulsed with 10 μM EdU (Invitrogen) for 24 hours post-LPS exposure and then chased with Complete Medium containing IL-5 for an additional 4 days. Following each chase, cells were fixed in 4% paraformaldehyde in 1×PBS at pH 7.4 and permeabilized with Click-iT™ saponin-based permeabilization and wash reagent (Invitrogen) stained with PE-Siglec-F (BD Bioscience) and the Click-iT™ EdU-AlexaFluor647 staining kit, per the manufacturer's instructions (Invitrogen).

### Apoptosis Detection

LDBM cells were collected and plated in 24-well plates, as previously described. For the assay, medium was changed after 8 or 14 days in culture to Complete Medium without cytokine or treatment, Complete Medium plus 10 ng/mL IL-5, or Complete Medium with 10 ng/mL IL-5 and 0.01 μg/mL, 0.1 μg/mL or 1 μg/mL LPS. Cells were harvested after 1, 6, or 24 hours and labeled with rat anti-mouse AnnexinV-APC (BD Bioscience), 7AAD (BD Bioscience) and CD34-FITC (Day 8 cells) or Siglec-F-PE (Day 14 cells).

### Results

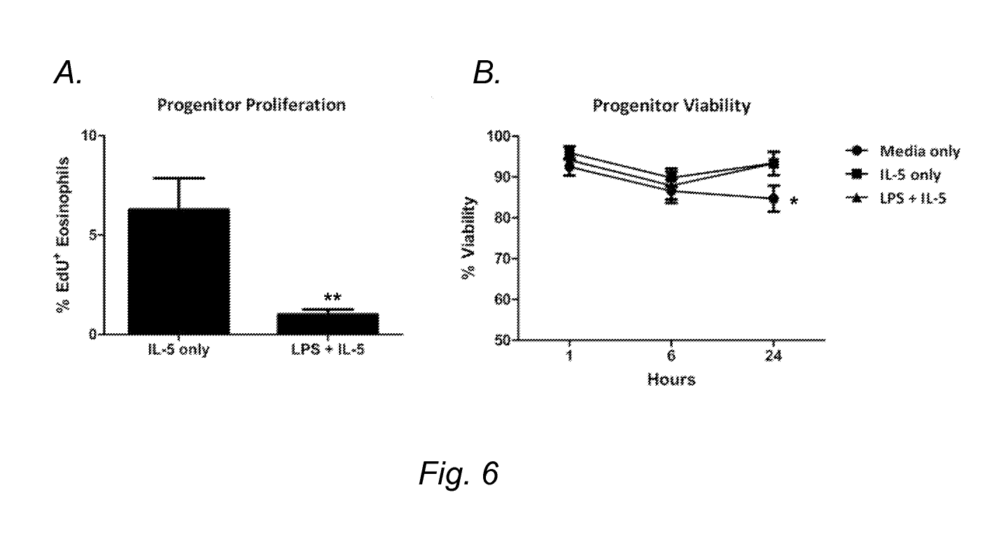

To mark proliferating progenitors, developing eosinophils were treated with LPS for 24 hours; LPS was then removed, and cells were then incubated with IL-5 along with EdU, a nucleoside analog of thymidine (5-ethynyl-2′-deoxyuridine), for 24 hours. The developing eosinophils were further cultured with IL-5 alone for another 3-4 days to complete differentiation into mature eosinophils. The percentage of developing eosinophils that were proliferating and incorporating EdU into their DNA after LPS exposure was found to be significantly reduced (85±7%, n=5 independent experiments) when compared to cells that were cultured in IL-5 alone (FIG. 6A).

The viability dyes annexin V and 7AAD were used to assess EoP viability with viable cells defined as annexin V-negative/7AAD-negative. After 24 hours, IL-5 alone had promoted EoP viability compared with medium alone, and LPS (1 μg/mL) stimulation for up to 24 hours had no effect on IL-5-mediated EoP survival (FIG. 6B). There was no change in the viability of developing eosinophils exposed to lower doses of LPS, and no change in the viability of mature eosinophils was detected after exposure to LPS. LPS exposure therefore suppresses eosinophil production via inhibition of progenitor cell proliferation and expansion rather than induction of apoptosis and cell death.

FIG. 6A. Cells were treated with IL-5 only or with IL-5 and LPS (LPS) for 24 hours, then pulsed with EdU for an additional 24 hours. Thereafter, cells were cultured for an additional 3-4 days with IL-5 alone. The percentage (mean SEM) of Siglec-F⁺EdU⁺ cells at day 14 is shown. **P=0.01, n=5 independent experiments.

FIG. 6B. The percentages (mean SEM) of viable cells in cultures with IL-5 only (squares), LPS with IL-5 (triangles), or with medium only with no added cytokines (circles) after 1, 6, and 24 hours starting with Day 8 cultured cells are shown. *P<0.05 with n=4 independent experiments.

### Example 7

### Reduction in the Number of EoP Colonies and Circulating Eosinophils by Endotoxemia

To determine if systemic LPS results in a reduction in the number of EoPs in vivo, endotoxemia was induced via intraperitoneal administration of LPS in wild-type mice, and bone marrow cells were harvested 24 hours later. EoP expansion was examined indirectly via eosinophil colony formation from harvested LDBM cells.

### Colony Forming Unit (CFU) Assay

CD2-IL-5 transgenic (Dent, et al. J. Exp. Med. 172:1425-31 (1990)) and BALB/c mice were injected intraperitoneally with 10 μg, 25 μg, or 40 μg of LPS (Sigma) in 100 μl 0.9% sodium chloride. Bone marrow was collected, and LDBM cells were isolated and resuspended in IMDM medium 24 hours after treatment, as described previously. Methocult medium (M3231; STEMCELL Technologies, Vancouver, BC) was prepared containing either 50 ng/mL murine IL-5 (Peprotech) or 50 ng/mL murine IL-5, 10 ng/mL granulocyte macrophage colony stimulating factor (GM-CSF) (Peprotech), and 20 ng/mL murine IL-3 (Peprotech). LDBM cells were then added at a concentration of 5×104 cells/mL (G-CSF cultures) or 1×105 cells/mL (cultures containing IL-5), and 1 mL of each culture was plated in 35-mm dishes in triplicate. Colonies on each plate were counted following 7-10 days and normalized to the number of cells plated in each dish. Each triplicate was averaged and reported as colonies formed (CFU) per 10,000 cells plated.

### Results

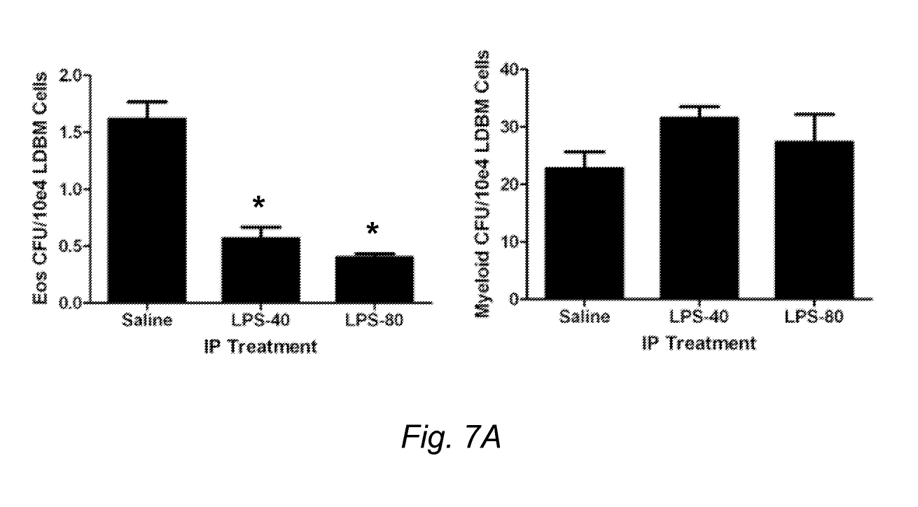

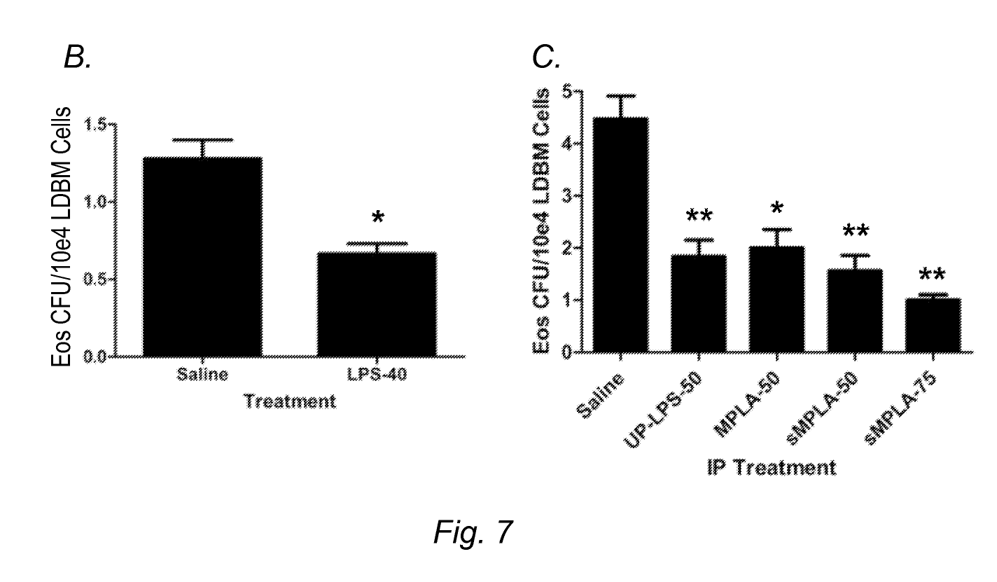

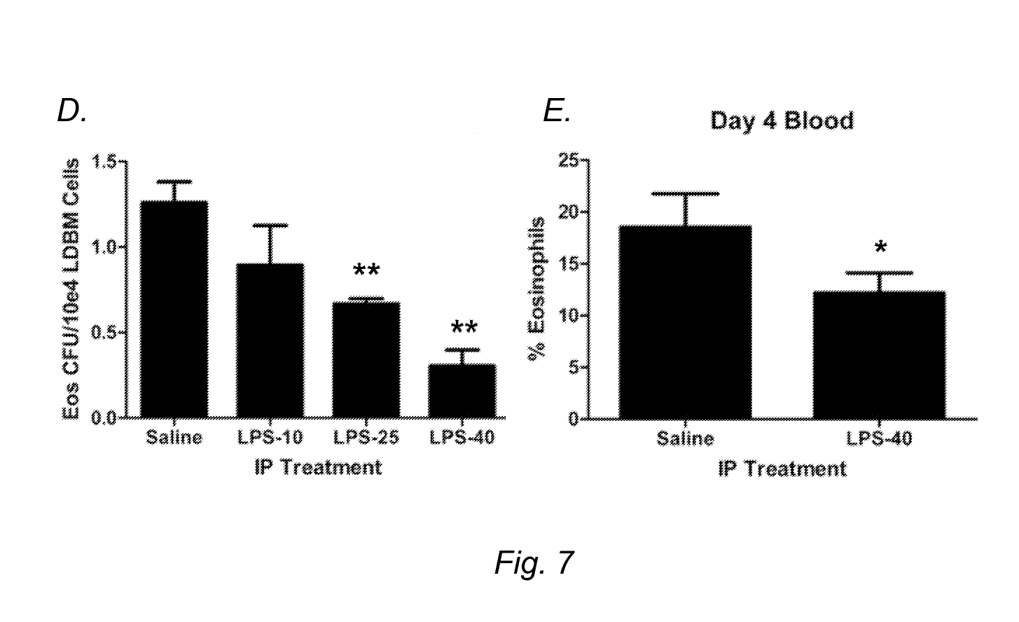

Endotoxemia resulted in a significant, dose-dependent reduction in the number of IL-5-induced eosinophil colonies formed from LDBM cells as compared to LDBM cells from saline-treated controls. In contrast, the number of mixed myeloid colonies containing neutrophils and macrophages was not significantly changed, indicating specificity of the effect of endotoxemia on eosinophils (FIG. 7A). The reduction in EoPs was still present more than one week after LPS treatment (FIG. 7B). The effect of TLR4 activation in vivo was confirmed as MPLA- and sMPLA-induced endotoxemia in wild-type mice resulted in a similar reduction in eosinophil colony formation as that induced by systemic LPS (FIG. 7C).

The ability of LPS to decrease EoPs in hypereosinophilic mice driven by the CD2-IL-5 transgene (Dent, et al. J. Exp. Med. 172:1425-31 (1990)) was then examined. Despite the presence of constitutive IL-5 expression in CD2-IL-5 transgene-driven hypereosinophilic mice, LPS-induced endotoxemia resulted in a dose-dependent decrease in EoP ex vivo expansion in this hypereosinophilic mouse model (FIG. 7D). Blood eosinophilia was significantly reduced 4 days after LPS treatment (FIG. 7E).

FIG. 7A. Eosinophil (Eos) and mixed myeloid colonies (mean SD) formed from LDBM cells harvested from wild-type mice 24 hours after intraperitoneal (IP) treatment with saline, 40 mg LPS (LPS-40), or 80 mg LPS (LPS-80) are shown. Results are representative of 3 independent experiments. *P<0.05.

FIG. 7B. Eosinophil colonies (mean SD) formed from LDBM cells harvested from wild-type mice 8 days after IP with saline or LPS-40 are shown. Results are representative of 3 independent experiments. *P<0.05.

FIG. 7C. Eosinophil colonies (mean SD) formed from LDBM cells harvested from wild-type mice 24 hours after IP with saline, 50 mg of ultrapure LPS (UP-LPS-50), 50 mg of MPLA (MPLA-50), or 50 or 70 mg synthetic MPLA (sMPLA-50 or sMPLA-70) are shown. Results are representative of 2 independent experiments. *P<0.05 and **P<0.01.

FIG. 7D. Eosinophil colonies (mean SEM, n=3 independent experiments) formed from LDBM cells harvested from IL-5 transgenic mice 24 hours after IP with saline or 10 mg, 25 mg, or 40 mg of LPS (LPS-10, LPS-25, LPS-40) are shown. **P<0.01.

FIG. 7E. Percentages of blood eosinophils in whole blood from IL-5 transgenic mice 4 days after IP with saline or 40 mg of LPS (LPS-40) are shown. Results are representative of 2 experiments. *P<0.05.

### Example 8

### Inhibition of Human EoPs by TLR4 Ligands

TLR4 ligands were demonstrated to suppress eosinophil production in vitro and in vivo in mice (Examples and 3, 6, and 7). Therefore, a subsequent study was designed to determine whether suppression of eosinophil production by TLR4 ligands is conserved between mice and humans.

### Human Eosinophil Liquid Culture System

CD34⁺ progenitors (>89% purity) from human umbilical cord blood cells were plated in Complete Medium supplemented with SCF and FLT3L at 50 ng/mL along with IL-3, IL-5, and GM-CSF at 10 ng/mL in 24-well plates at 1×105 cells/well for 6 days, with a half-medium change on day 3. On day 6, cells were pooled, counted, and seeded in Complete Medium with IL-3 and IL-5 at 10 ng/mL in 6-well plates at 1×106 cells/mL, with a half-medium change on day 9. On day 12, medium was changed to Complete Medium containing IL-5 at 10 ng/mL alone or IL-5 and LPS (Sigma) at 1 μg/mL. Half-medium changes were performed every 3 days thereafter. For cells exposed to LPS, medium was replaced on day 13 to Complete Medium with IL-5, with half-medium changes performed every 3 days thereafter. Cells were counted on day 21 with at least 4 wells counted per experiment. Culture yields were >95% eosinophil peroxidase-positive cells after day 21.

### Results

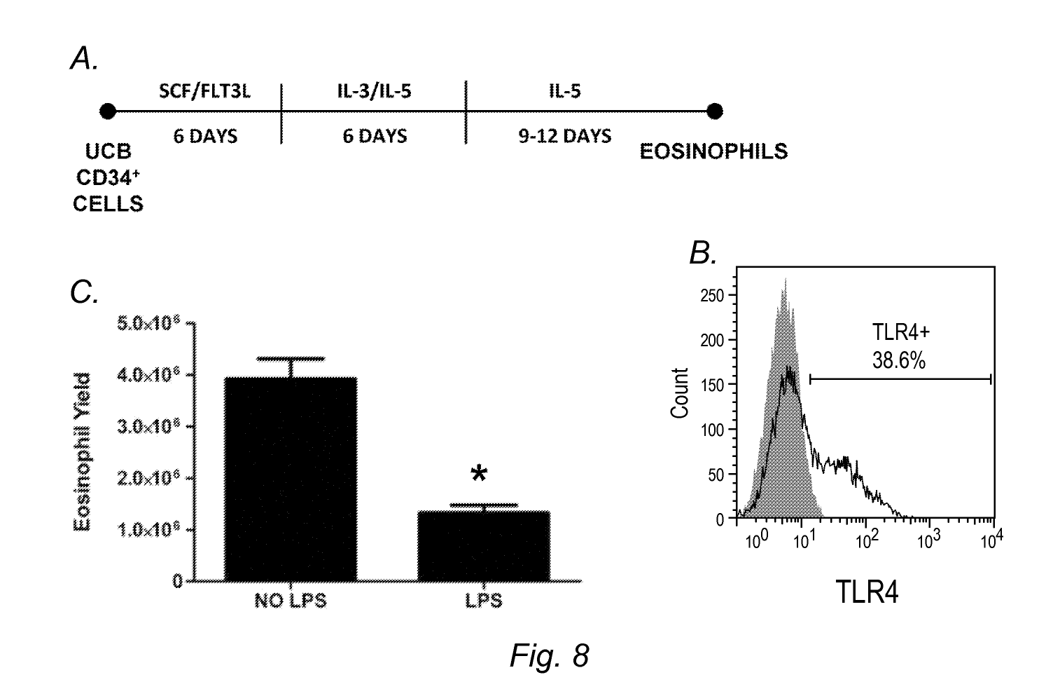

Eosinophil development from purified CD34⁺ progenitors derived from human cord blood was examined (FIG. 8A). Surface expression of TLR4 was detected on progenitors following stimulation with SCF and FLT3L for 6 days (FIG. 8B). Developing human eosinophils were then treated from day 12 of culture with IL-5 and LPS for 24 hours, followed by continued eosinophil differentiation by culture in IL-5 for 8 days. Similar to what was observed with murine eosinophils cultured from bone marrow cells, the total yield of human eosinophils cultured from human CD34⁺ progenitors was significantly reduced following LPS stimulation (FIG. 8C).

FIG. 8A. A schematic of the human eosinophil liquid culture system is depicted. CD34′ purified human umbilical cord blood (UCB) progenitors are cultured in medium containing the cytokines SCF and FLT3L for 6 days to expand the progenitor population, after which the cells are cultured in medium supplemented with IL-3 and IL-5 for 6 days and then in medium with IL-5 alone for an additional 9-12 days to yield mature eosinophils.

FIG. 8B. A representative histogram showing TLR4 expression on progenitors after 6 days of SCF and FLT3L stimulation (n=2 experiments) is depicted. Isotype control antibody staining is represented by the filled gray line.

FIG. 8C. Eosinophil yields at day 21 with and without LPS (1 mg/mL) stimulation are shown (mean SD, n=4 wells per experiment). Results are representative of 2 experiments. ^{&}P<0.0001 when compared to yield with no LPS exposure.

### Example 9

### Effect of LPS on Developing Eosinophils

As surface TLR4 was detected on the progenitors in response to IL-5 but not on the surface of the mature CCR3-expressing eosinophils, the eosinophil lineage therefore has distinct sensitivity to TLR4 activation, depending on differentiation state (Example 2). The TLR4-expressing cells were confirmed to be EoPs as the TLR4 cells isolated from the cultures differentiated into a pure eosinophil culture (Example 2).

A striking LPS-mediated reduction in the production of eosinophils from EoPs was observed in vitro; however, EoP differentiation was not inhibited, as the percentage of cells with surface Siglec-F expression and the level of Siglec-F expression were similar between cells regardless of LPS exposure (Example 6). Pulse-chase experiments revealed a marked decrease in the number of cells that were proliferating after LPS stimulation (Example 6).

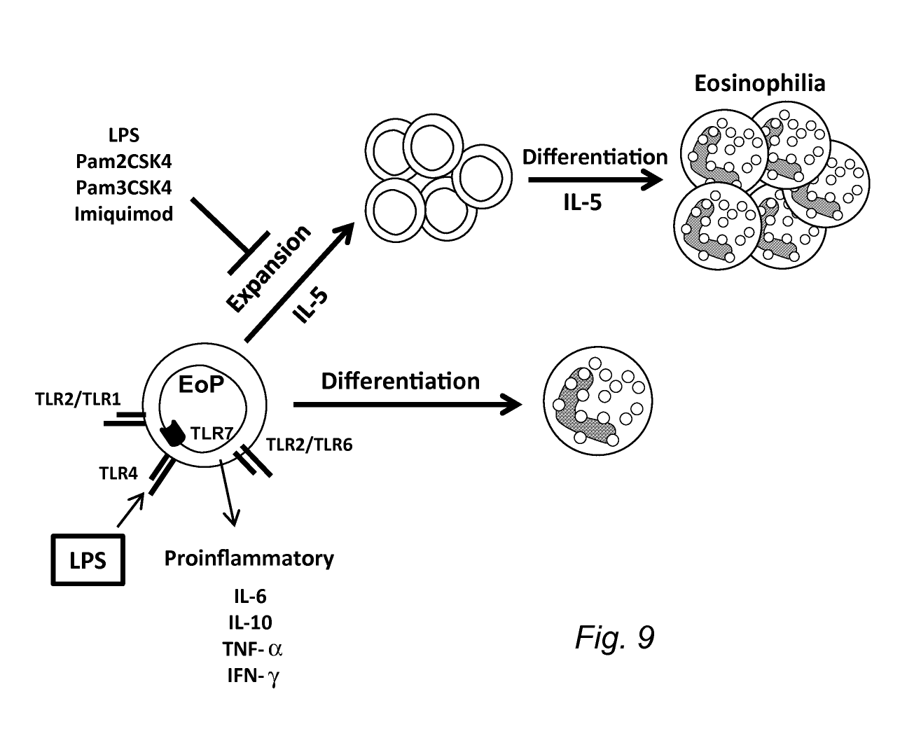

The suppressed proliferation was not secondary to IFN-γ, IL-10, TNF-α, or IL-6, as addition of these cytokines individually or as a cocktail (as well as the addition of anti-IFN-γ) to the cultures had no significant effect on the yield of mature eosinophils (Example 5). No increase in apoptosis or cell death of EoPs or mature eosinophils was detected following LPS stimulation (Example 6). Therefore, LPS has a direct effect on developing eosinophils, mediated via TLR4 activation and resulting in suppression of the EoP proliferative response to IL-5 (FIG. 9). Similar activity was also demonstrated with another TLR4 agonist MPLA (Examples 3 and 7).

FIG. 9. EoPs respond to the environment (e.g. bacterial and viral infection) via activation of TLRs that results in suppression of EoP expansion and ultimately in inhibition of IL-5-mediated eosinophilia. In addition, EoPs respond to LPS with production of the pro-inflammatory cytokines IL-6, IL-10, TNF-α, and IFN-γ, resulting in the ability to influence the local microenvironment in the bone marrow.

### Example 10

### Treatment of Eosinophilia-Associated Conditions Via Administration of a TLR4, TLR2, or TLR7 Ligand

The results described herein demonstrate the mechanism of sepsis-associated eosinopenia and provide a heretofore unknown therapeutic strategy to target EoPs to inhibit eosinophil production. These studies demonstrate a significant reduction in eosinophil yield with even a short-term, low-dose exposure to the TLR4 ligand LPS.

Under conditions (IL-5 transgene expression) that result in hypereosinophilia in mice, LPS was a potent inhibitor of eosinophil production (Example 6). Patients with chronic Strongyloides infection can initially have elevated blood eosinophil counts that then markedly drop with the bacteremia that can accompany hyperinfection (Newberry, et al. Chest 128:3681-4 (2005); Fardet, et al. J. Infect. 54:18-27 (2007)).

As progress has been made in understanding TLR biology and the role of TLRs in human disease, the interest in developing TLR modulators for therapy has also increased (Hennessey, et al. Nat. Rev. Drug Discov. 9:293-307 (2010)). TLR2 and TLR4 agonists have been shown to have strong adjuvant and anti-tumor activity in vivo (D'Agostini, et al. Int. Immunopharmacol. 5:1205-12 (2005); Murata Cancer Sci. 99:1435-40 (2008)). The TLR4 agonist MPLA has been approved in Europe as a vaccine adjuvant and is currently a component in vaccines directed against hepatitis B and human papillomavirus (Garcon, et al. BioDrugs. 25:217-26 (2011); Vandepapeliere, et al. Vaccine 26:1375-86 (2008)). TLR agonists are also being considered for their therapeutic potential in allergy and asthma (Broide Annu. Rev. Med. 60:279-91 (2009)). For instance, MPLA is being evaluated as a component in the vaccine Pollinex Quattro directed against ragweed pollen extract (Baldrick, et al. J. Appl. Toxicol. 27:399-409 (2007)).

Developing eosinophils demonstrated a functional response to LPS by producing a number of inflammatory cytokines. Therefore, EoPs can influence hematopoiesis of other lineages via secretion of these potent cytokines into the local microenvironment following TLR activation. Both IFN-γ and TNF-α have been shown to have suppressive effects on early progenitors (Sharma, et al. J. Biol. Chem. 286:27506-14 (2011)); however, the results described herein demonstrated that they had no effect on the eosinophil lineage-committed precursors, suggesting differential sensitivity to these inflammatory cytokines after lineage commitment. Murine eosinophils have recently been shown to have an important role in the long-term survival of plasma cells in the bone marrow via secretion of IL-6 and APRIL (Chu, et al. Nat. Immunol. 12:151-9 (2011)). LPS-stimulated EoPs and mature eosinophils can therefore promote persistent production of immunoglobulins via enhanced secretion of these cytokines. Recent evidence indicates that eosinophils are important regulators of local immune microenvironments under homeostatic and disease states (Lee, et al. Clin. Exp. Allergy 40:563-75 (2010); Akuthota, et al. J. Innate Immun. 3:113-9 (2011); Jacobsen, et al. J. Allergy Clin. Immunol. 119:1313-20 (2007); Allakhverdi, et al. J. Allergy Clin. Immunol. 123:472-8 (2009)). The data presented herein demonstrate the importance of EoPs in regulating the bone marrow microenvironment via secretion of cytokines, and potentially other mediators, in response to bacterial infections (FIG. 9).

Heretofore, there have been no approved drugs that selectively target eosinophils, as therapeutic development for eosinophil-associated disorders has focused exclusively on blocking IL-5/IL-5Rα activity via neutralizing monoclonal antibodies (Ogbogu, et al. J. Allergy Clin. Immunol. 124:1319-25 (2009); Rothenberg, et al. N. Engl. J. Med. 358:1215-28 (2008)). While anti-IL-5-targeted therapy has been successful in decreasing blood and tissue eosinophilia in patients, it has no effect on the increased numbers of EoPs in the bone marrow of patients (Menzies-Gow, et al. J. Allergy Clin. Immunol. 111:714-9 (2003)), making them theoretically susceptible to rebound eosinophilia upon withdrawal of treatment. Recent data has emerged that IL-5Rα can be up-regulated by anti-IL-5 treatment and can be responsible for the rebound eosinophilia observed after anti-IL-5 therapy in humans (Kim, et al. J. Allergy Clin. Immunol. 114:1449-55 (2004); Stein, et al. J. Allergy Clin. Immunol. 121:1473-83 (2008)). This process may limit the usefulness of anti-IL-5 therapies and has significant implications for treated patients.

As described in the examples above, EoPs represent a valuable therapeutic target that can result in long-lasting eosinophil depletion either alone or in combination with anti-IL-5 therapeutics. These findings provide evidence for the importance of innate immune receptors on the eosinophil lineage-committed progenitors during development and thus substantiate a broad role and mechanism for TLR modulators in inflammatory diseases.

The data presented herein demonstrate that stimulating TLR2, TLR4 and TLR7 pathways can lead to diminished eosinophil production. Therefore, emerging compounds that activate these pathways can be used therapeutically to treat eosinophilic disorders (FIG. 9).

A subject is diagnosed as having an eosinophilia-associated condition. A TLR4 ligand, such as LPS, or a TLR2 or TLR7 ligand is administered to the subject. Following administration, eosinophil progenitor proliferation in the subject is suppressed, resulting in reduced eosinophil production and reduced peripheral eosinophilia, thereby alleviating symptoms associated with the eosinophilia-associated condition.

### Example 11

### EoPs in Bacterial Infection

Severe sepsis is a leading cause of mortality in the ICU (Angus, et al. Crit. Care Med. 29:1303-10 (2001)). Gram-negative bacterial pathogens are the predominant causative microorganisms of severe ICU infections (Vincent, et al. JAMA 302:2323-9 (2009)). A marked reduction in circulating eosinophils has long been associated with bacterial infection and has been shown to be a reliable marker of systemic infection upon admission to the ICU (Abidi, et al. Crit. Care 12:R59 (2008); Shaaban, et al. J. Crit. Care 25:570-5 (2010)).

Eosinophils have been shown to have anti-bacterial properties and be beneficial in a polymicrobial sepsis model (Linch, et al. Infect. Immun. 77:4976-82 (2009); Yousefi, et al. Nat. Med. 14:949-53 (2008); Linch, et al. Endocr. Metab. Immune Disord. Drug Targets 11:165-72 (2011)). However, the striking reduction in EoP cell proliferation after exposure to TLR4 ligands, as described herein, can benefit the host via the reduced energy and raw material consumption by the eosinophil lineage. It is heretofore unknown whether inhibition of eosinophil production by microbial products is beneficial to the survival of specific microbial species. EoPs can respond to a number of different viral and bacterial systemic infections, as expression of six different TLR mRNAs during eosinophil differentiation was observed.

Data for all of the above experiments were analyzed using either a 2-tailed unpaired Student's t test or a one-way ANOVA with Bonferroni post-hoc test as appropriate (GraphPad Prism). Differences were considered statistically significant when P<0.05. Data is presented as mean±SEM except for representative experiments for which data is presented as mean±SD.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method of treating an eosinophilia-associated condition in a subject in need thereof, comprising:
identifying a subject with an eosinophilia-associated condition; and administering to the subject a suppressor of eosinophil progenitor (EoP) proliferation, wherein administration of the suppressor results in treatment of the eosinophilia-associated condition.

2. The method of claim 1, wherein the suppressor interacts with a Toll-like receptor.

3. The method of claim 2, wherein the Toll-like receptor is at least one of TLR-4, a TLR-2 heterodimer, or TLR-7.

4. The method of claim 2, wherein the Toll-like receptor is TLR-4 and wherein the suppressor is selected from a lipopolysaccharide (LPS), monophosphoryl lipid A (MPLA), mannan, a phospholipid, MMTV, RSV, ultrapure MPLA, synthetic MPLA (sMPLA), HSP-22, HSP-60, HSP-70, HSP-96, fibrinogen (extra domain A), minimally modified low-density lipoprotein, and surfactant protein A.

5. The method of claim 2, wherein the Toll-like receptor is a TLR-2 heterodimer and wherein the suppressor is selected from Pam2CSK4, Pam3CSK4, lipoteichoic acid, zymosan, a porin, MALP2, a bacterial peptidoglycan, lipoarabinomannan, HSP-60, HSP-70, HSP-96, and HMGB1.

6. The method of claim 2, wherein the Toll-like receptor is TLR-7 and wherein the suppressor is selected from CL264, imiquimod, a viral ssRNA, a GU-rich oligoribonucleotide, loxoribin, resiquimod, an adenosine derivative a guanosine derivative, and an immune-complex ssRNA.

7. The method of claim 2, wherein the suppressor is selected from a lipopolysaccharide (LPS), monophosphoryl lipid A (MPLA), mannan, a phospholipid, MMTV, RSV, ultrapure MPLA, synthetic MPLA (sMPLA), HSP-22, HSP-60, HSP-70, HSP-96, fibrinogen (extra domain A), minimally modified low-density lipoprotein, surfactant protein A, Pam2CSK4, Pam3CSK4, lipoteichoic acid, zymosan, a porin, MALP2, a bacterial peptidoglycan, lipoarabinomannan, HSP-60, HSP-70, HSP-96, HMGB1, CL264, imiquimod, a viral ssRNA, a GU-rich oligoribonucleotide, loxoribin, resiquimod, an adenosine derivative a guanosine derivative, and an immune-complex ssRNA.

8. The method of claim 1, wherein the eosinophilia-associated condition is selected from an eosinophil-associated gastrointestinal disorder, eosinophilic pneumonia, allergies, asthma, atopic dermatitis, drug hypersensitivity, eosinophilic leukemia, Churg-Strauss syndrome, and hypereosinophilic syndrome.

9. The method of claim 1, wherein administration of the suppressor results in reduced eosinophil production.

10. The method of claim 1, wherein administration of the suppressor results in reduced peripheral eosinophilia.
